(19) United States
(12) Patent Application Publication
(10) Pub. No.: US 2024/0066282 A1
(43) Pub. Date: Feb. 29, 2024

(54) PASSIVE TEMPERATURE LOGGING FOR AN OBJECT

(71) Applicant: Stryker Corporation, Kalamazoo, MI (US)

(72) Inventor: Fred W. Chapman, Newcastle, WA (US)

(73) Assignee: Stryker Corporation, Kalamazoo, MI (US)

(21) Appl. No.: 18/457,903

(22) Filed: Aug. 29, 2023

Related U.S. Application Data
(60) Provisional application No. 63/402,159, filed on Aug. 30, 2022.

(51) Int. Cl. A61N 1/04; A61N 1/39

(52) U.S. Cl. CPC A61N 1/0408; A61N 1/3904

(57) ABSTRACT

Passively logging the temperature, among other environmental conditions, of an object is described. A passive temperature-logging device may be coupled to an object. The device may include a temperature sensor(s) for sensing a temperature of the object. In some examples, the passive temperature-logging device, and/or the object itself, may further include a memory(ies) and/or an output device(s). The memory is configured to store temperature data representing a history of temperatures sensed by the temperature sensor, and the output device is configured to provide an output based on the temperature data.

### CROSS-REFERENCE TO RELATED APPLICATION

This application claims the benefit of U.S. Provisional Application No. 63/402,159, titled “PASSIVE TEMPERATURE LOGGING FOR AN OBJECT”, and filed on Aug. 30, 2022, which is incorporated by reference herein in its entirety.

### BACKGROUND

Medical devices may be suitable for use, and/or storage, in particular environmental conditions, such as within a prescribed temperature range. For example, defibrillation electrodes should be stored within a prescribed temperature range to extend their shelf life. As another example, external defibrillators, such as automated external defibrillators (AEDs), have a limited temperature range in which they can function.

### BRIEF DESCRIPTION OF THE DRAWINGS

FIG. 1 illustrates an example system including a passive temperature-logging device configured to passively log the temperature of an object, according to the techniques described herein.

FIG. 2 illustrates example components of a passive temperature-logging device, according to the techniques described herein.

FIG. 3 illustrates an example passive temperature-logging device coupled to a packaged set of electrodes, the passive temperature-logging device configured to passively log the temperature of the packaged electrodes, according to the techniques described herein.

FIG. 4 illustrates an example external defibrillator stored in a case, the case including a component that is configured to interrogate a passive temperature-logging device coupled to an external defibrillator that is stored within the case, according to the techniques described herein.

FIG. 5 illustrates example components of an external device configured to receive, from a passive temperature-logging device, temperature data representing a temperature history of an object to which the passive temperature-logging device is coupled, according to the techniques described herein.

FIG. 6 illustrates an example external defibrillator that is configured to receive temperature data from a passive temperature-logging device coupled to a set of electrodes and to output an indication of a usability of the electrodes based on the received temperature data, according to the techniques described herein.

FIG. 7 illustrates an example external defibrillator configured to output an indication of a usability of the external defibrillator based on a temperature history of the external defibrillator obtained by a passive temperature-logging device coupled to the external defibrillator, according to the techniques described herein.

FIG. 8 illustrates an example process for passively logging the temperature of an object and providing an output based on the passively-logged temperature, according to the techniques described herein.

FIG. 9 illustrates an example process for adjusting a temperature-sensing frequency of a passive temperature-logging device based on an amount of harvested energy that is currently stored by the passive temperature-logging device, according to the techniques described herein.

FIG. 10 illustrates an example process for passively logging the temperature of an object and sending corresponding temperature data to an external device in an energy-efficient manner, according to the techniques described herein.

FIG. 11 illustrates an example process for analyzing a temperature history of an object and providing an output based on the temperature history analysis, according to the techniques described herein.

FIG. 12 illustrates an example process for adjusting an interrogation frequency of an external device based on an amount of energy available to a passive temperature-logging device, according to the techniques described herein.

### DETAILED DESCRIPTION

Various objects may be adversely impacted when they are exposed to certain environmental conditions. Defibrillation electrodes, as one example, have a finite shelf life to ensure that the electrodes will perform adequately when they are used with a defibrillator to administer defibrillation therapy to a patient, and this shelf life is dependent on the storage temperature of the electrodes. For instance, storage temperatures that are too hot can accelerate the rate at which the electrode gel dries out, thereby causing a degradation in performance of the electrodes and shortening the shelf life of the electrodes as a consequence. Without knowing how end users will store electrodes, the shelf life of electrodes is oftentimes spec'd for the worst-case storage conditions, leading to a spec'd shelf life that is shorter than the actual, useful life of many electrodes in the field. In other words, an end user may discard electrodes before they need to be discarded. As another example, external defibrillators, such as AEDs, have a limited temperature range in which they can function. For instance, an AED can become unusable if it is stored at temperatures that are too cold. End users are advised to store AEDs within a prescribed temperature range (e.g., within a heated case, or in a warm place, if, say, the AED is stored at a location with a particularly cold climate, such as a ski resort). However, end users do not always store AEDs within the recommended temperature range. As a result, a number of AEDs in the field may be unable to administer defibrillation therapy when they are needed to save someone's life.

Moreover, many objects do not have a power supply, and certain objects that do have a power supply (e.g., a battery) may be configured to use that limited power supply to perform critical, life-saving functions, making active environmental parameter monitoring of such objects impracticable. For example, the battery power of an external defibrillator should be reserved to carry out the critical, life-saving function of administering defibrillation therapy when the defibrillator is separated from mains electricity; not for actively monitoring the temperature of the defibrillator.

This disclosure provides systems, devices, and methods for passively logging the temperature, among other environmental conditions, of an object. A passive temperature-logging device (sometimes referred to herein as a “device”) may be coupled to (e.g., built into, removably coupled to, etc.) an object. The passive temperature-logging device may include a temperature sensor(s) for sensing a temperature of the object. In some examples, the passive temperature-logging device, and/or the object itself, may further include a memory(ies) and/or an output device(s). The memory is configured to store temperature data representing a history of temperatures sensed by the temperature sensor, and the output device is configured to provide an output based on the temperature data, as described herein.

In some examples, the temperature sensor is configured to passively sense the temperature of the object using energy harvested from a non-battery energy source. For example, the passive temperature-logging device may include an energy harvesting component configured to harvest energy from a non-battery energy source when the object is separated from mains electricity. To illustrate, the object may be a set of electrodes for an external defibrillator. The electrodes may not have a power supply, and, when the electrodes are decoupled from the external defibrillator, both mains electricity and the battery of the external defibrillator are unavailable to the electrodes. Accordingly, an energy harvesting component of the passive temperature-logging device may be used to harvest energy from a non-battery energy source, and this harvested energy may be used to power one or more components of the passive temperature-logging device (and/or to power one or more components of the object), such as the temperature sensor(s), the memory(ies), and/or the output device(s). Various examples of energy harvesting components are described herein, such as an antenna(s) configured to harvest energy from electromagnetic (EM) radiation in a particular spectrum(s), a photovoltaic cell(s) configured to harvest energy from sunlight and/or artificial light, a mechanical motion device(s) configured to harvest energy from motion of a mass, etc. In some examples, in addition to, or in lieu of, utilizing an energy harvesting component, the temperature sensor of the passive temperature-logging device may be configured to sense a temperature of the object (e.g., the set of electrodes) by detecting a change in voltage across a diode. For instance, the temperature sensor may represent a passive radio frequency identification (RFID) temperature sensor, which may not require any energy to function as a passive temperature sensor.

The techniques, devices, and systems described herein allow for obtaining a precise, historical record of the environmental condition(s) (e.g., temperatures) of an object that does not have, or does not have access to, a power supply. The techniques, devices, and systems described herein also allow for obtaining such a historical record of the environmental condition(s) (e.g., temperatures) of a battery-powered object without depleting the object's battery. This provides an entity, such as a manufacturer or a vendor of the object, insight into how the object has been, or is being, stored and/or transported (e.g., in terms of the temperatures to which the object was exposed over time).

If the object is a set of electrodes (e.g., unopened (packaged) electrodes), the techniques, device, and systems described herein allow for obtaining a record of the temperature history of the electrodes while keeping the electrodes packaged (e.g., in a hermetically-sealed container (e.g., pouch)) to maintain a level of humidity inside of the sealed container, thereby maximizing the shelf life of the electrodes while passively logging the temperature of the electrodes. Knowledge of the temperature history of the electrodes allows an end user to avoid prematurely discarding functional electrodes. Alternatively, a user may discard electrodes at an appropriate time if, say, the electrodes have been improperly stored (e.g., at temperatures that are too hot).

If the object is an external defibrillator, the techniques, device, and systems described herein allow for obtaining a record of the temperature history of the external defibrillator without depleting the charge of the defibrillator's battery. Accordingly, the external defibrillator can remain ready to administer defibrillation therapy, seeing as how its battery power is reserved for performing critical, life-saving functions as opposed to being used to actively log the temperature of the external defibrillator over time.

In some examples, the output device(s) is a transceiver, and providing the output includes sending the temperature data to an external device(s). Accordingly, the external device can receive the temperature data (or a result determined therefrom) from the passive temperature-logging device, and the external device can analyze the temperature history of the object. In some examples, the external device is configured to output an indication of a usability of the object as a result of analyzing the temperature history, such as a warning or a notification that the object should be discarded. In some examples, the external device is configured to predict a time remaining until the object is expired using a result of analyzing the temperature history. If the object is a set of electrodes, for example, the external device may dynamically predict (e.g., update) a time remaining until the set of electrodes is expired, and the external device may output an indication of this time remaining via an output device(s) of the external device. For instance, if the external device is an external defibrillator, the external defibrillator may display an updated expiration date of the electrodes and/or some other indication (e.g., a warning, notification, etc.) based on the predicted shelf life of the electrodes.

In some examples, the external device is configured to determine an amount of time that a temperature of the object was outside of a predetermined temperature range, and to send the amount of time to a remote asset management system configured to manage a fleet of objects. For example, if the object is an external defibrillator, the external device may receive, from a passive temperature-logging device, temperature data representing a temperature history of the external defibrillator, determine, by analyzing the temperature history, an amount of time (e.g., a percentage of a period of time) that the temperature of the defibrillator was outside of a predetermined temperature range (e.g., below the temperature range), and send the amount of time to an asset management system. This information may be used to help an owner/manager of a fleet of defibrillators identify inappropriate storage situations so that those storage conditions can be changed, such as by sending a message(s) to the end user(s) in possession of the defibrillator(s).

Although many of the examples described herein pertain to an object that is a medical device (e.g., a set of electrodes, an external defibrillator, etc.), it is to be appreciated that the techniques, devices, and systems described herein may be implemented with respect to objects that are not classified as medical devices, such as food products, consumer electronic devices, or the like. Accordingly, it is to be appreciated that this disclosure is not limited to passively monitoring the temperature, among other environmental conditions, of medical devices. That is, similar advantages may be realized through passively logging the temperature, among other environmental conditions, of objects outside of the medical/healthcare industry that are either battery-powered or without power supplies. Furthermore, although many of the examples described herein pertain to passively sensing and/or logging the temperature of an object, other environmental conditions or parameters associated with the object may be passively sensed and/or logged in a similar manner to provide various benefits, as described herein. These and other technical benefits are readily appreciated in light of this disclosure, with detailed reference to the figures provided below.

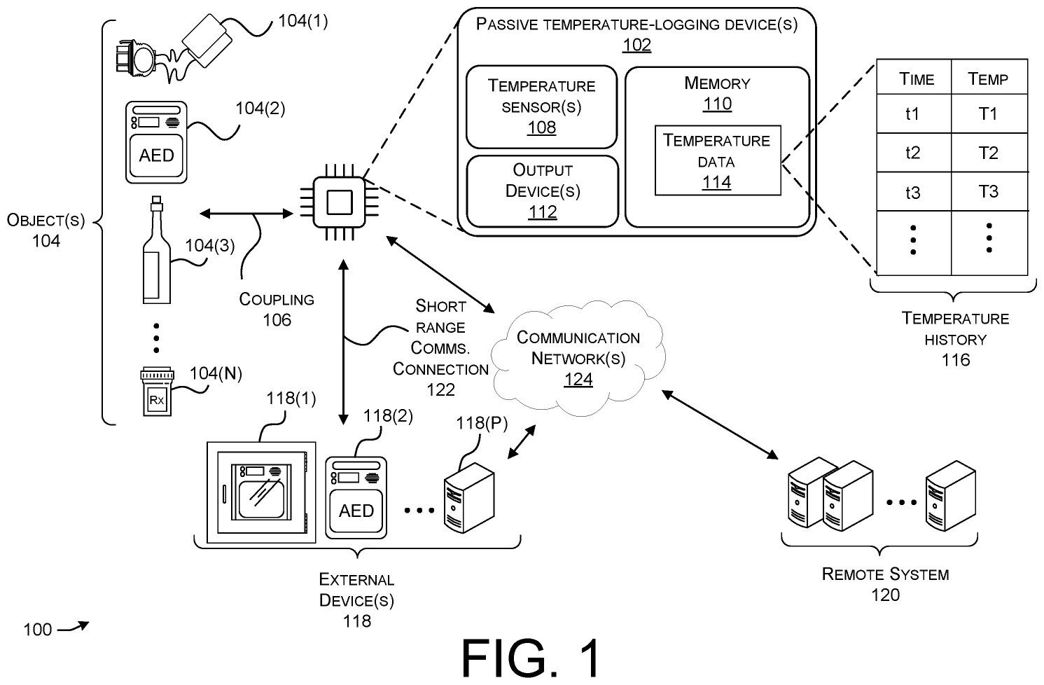

FIG. 1 illustrates an example system 100 including a passive temperature-logging device 102 (sometimes referred to herein as a “device 102”) configured to passively log the temperature, among other environmental conditions, of an object 104, according to the techniques described herein. FIG. 1 illustrates several examples of objects 104 whose temperature can be passively logged using the device 102, the example objects 104 including a set of electrodes 104(1) (e.g., defibrillation electrodes for an external defibrillator), an external defibrillator 104(2) (e.g., an AED), a food product 104(3) (e.g., a bottle of wine), and/or pharmaceuticals 104(N) (e.g., a bottle of prescription pills). It is to be appreciated that these example types of objects 104(1)-(N) are non-limiting examples, and that other types of objects 104 are contemplated, such as consumer electronic devices or other types of non-medical electronic devices, automotive parts (e.g., brake pads of an automotive vehicle), and the like. In some examples, the object 104 can be a living organism (e.g., a human, an animal, a plant, etc.) whose temperature, or another environmental condition(s), is passively logged over time using the device 102.

In some examples, the object 104 does not have, or does not have access to, a power supply. For example, the electrodes 104(1) may be configured to be used with (e.g., plugged into) a medical device, such as an external defibrillator, and to receive power from the external defibrillator when the electrodes 104(1) are plugged into the medical device. As such, the electrodes 104(1) may not have a battery, and when the electrodes 104(1) are separated from the medical device (e.g., the external defibrillator), the electrodes 104(1) do not have access to the battery the medical device, or to mains electricity for that matter (e.g., assuming the medical device can be plugged into a power outlet). As another example, food products 104(3) do not have a power supply. This is also the case with pharmaceuticals 104(N).

In some examples, the object 104 has a power supply, such as a battery, but the energy available from the power supply may be limited and/or reserved for performing critical functions, which, in some cases, may be life-saving functions. An external defibrillator 104(2), for example, may include a battery to allow for administering defibrillation therapy to a patient outside of a hospital setting, such as at a remote geographical location (e.g., on a mountain), in an ambulance, or the like. In this example, the external defibrillator does not have access to mains electricity (e.g., grid power accessible via a power outlet), and the battery power of the external defibrillator 104(2) should otherwise be reserved for such critical, life-saving functions in the absence of mains electricity.

FIG. 1 illustrates the device 102 as being coupled to the object 104 via a coupling 106. As used herein, the term “couple” may refer to an indirect coupling or a direct coupling between elements. The term “couple,” as used herein, may also refer to a removable coupling or a permanent coupling between the elements. Elements are removably coupled if a user or another entity is able to decouple the elements. Elements are permanently coupled if a user or another entity is unable to decouple the elements without destroying or significantly damaging the elements, or without undue effort to disassemble the elements using tools or machinery. As used herein, the term “couple” can be interpreted as connect, attach, affix, join, engage, interface, link, fasten, or bind. Unless otherwise specified herein, the term “couple” is to be interpreted as coupling elements in a mechanical sense, rather than in an electrical sense, for example. Nevertheless, it is to be appreciated that a mechanical coupling of elements may result in an electrical coupling(s) between multiple elements of a system. In some examples, the device 102 may be coupled to the object 104 by being built into, integrated with, and/or implanted in the object 104. For example, at a time of manufacturing the electrodes 104(1) and/or the external defibrillator 104(2), the passive temperature-logging device 102 may be built into and/or integrated in the electrodes 104(1) and/or the external defibrillator 104(2), such as by mounting the device 102 to a printed circuit board (PCB) within a housing of the external defibrillator 104(2). In other examples, the device 102 may be coupled to the object 104 after the object 104 is manufactured. If the object 104 is a living organism, the device 102 may be coupled to the object 104 by being implanted in, attached to, and/or worn by the living organism (e.g., a device 102 may be in the form of a patch, a wearable device, and/or an implantable device).

As shown in FIG. 1, the device 102 may include a temperature sensor(s) 108, a memory(ies) 110, and/or an output device(s) 112. In some examples, the object 104 itself may include at least some of these components. For example, the external defibrillator 104(2) may have a memory, as well as an output device(s) (e.g., a speaker(s), a display(s), a transceiver(s), etc.). Accordingly, if the passive temperature-logging device 102 is coupled to (e.g., built into) such an object 104, the device 102 may include the temperature sensor(s) 108, for example, and may utilize the memory and the output device of the object 104 in order to implement the functionality described herein.

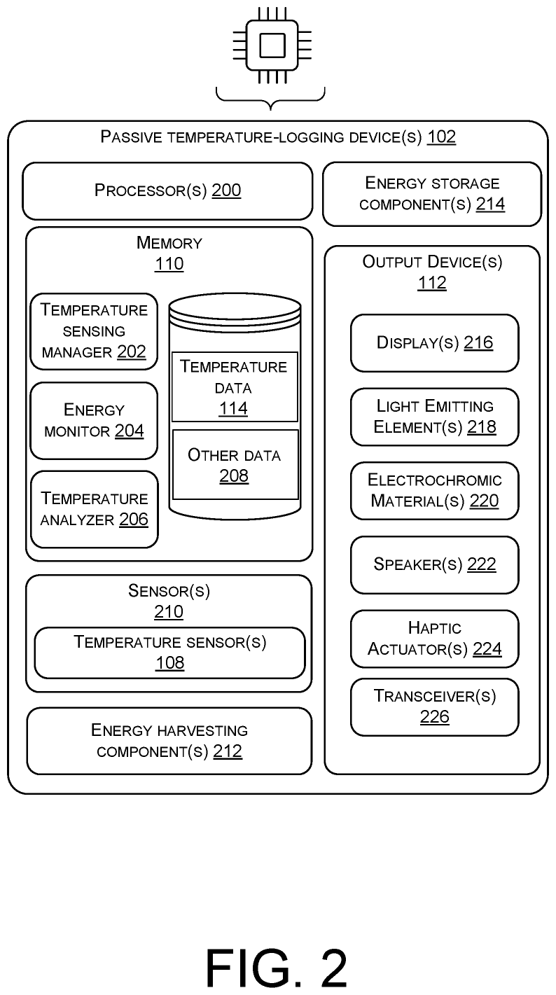

The temperature sensor 108 is configured to passively sense a temperature of the object 104. “Passive” temperature sensing, in this context, means that the temperature of the object 104 can be sensed without relying on a conventional power source (e.g., mains electricity or a battery, such as a primary battery of the object 104) to do so. In some examples, the temperature sensor 108 uses energy for sensing the temperature of the object 104, but the temperature sensing is nevertheless “passive,” as used herein, if the energy used for sensing the temperature is obtained from a non-battery energy source, which is sometimes referred to herein as an “ambient energy source.” In other examples, the temperature sensor 108 may not require any energy to function as a passive temperature sensor. For example, a change in temperature of the object 104 (or the object's surroundings) may cause a corresponding change in a monitored parameter. To illustrate, the temperature sensor 108 may represent a passive RFID temperature sensor that is configured to sense a temperature of the object 104 by detecting a change in voltage across a diode caused by a change in temperature of the object 104 and/or the object's surroundings. In other words, the change in voltage can be used as a proxy for determining a change in temperature, and if a reference temperature is known, the change in temperature relative to the reference temperature can be used to determine the temperature of the object 104. Examples of passive temperature sensing and passive temperature logging techniques are discussed in more detail with respect to FIG. 2, below.

The memory 110 is configured to store temperature data 114 representing a history of temperatures sensed by the temperature sensor 108. FIG. 1 illustrates an example temperature history 116 of an object 104 exhibited in the temperature data 114 stored in the memory 110. For example, the temperature history 116 may include a series of time values and corresponding temperature values that reflect a record of the temperatures to which the object 104 has been exposed over time. As will be described in more detail herein, the temperature sensing frequency may be a predetermined, regular frequency. Alternatively, the temperature sensing frequency may vary over time based on rules, and/or the temperature sensing frequency may be dynamically determined in real-time or near real-time based on the occurrence of events. As such, the difference between sequential temperature measurements (e.g., the time difference between times t1 and t2 in FIG. 1) may be on the order of seconds, minutes, hours, or any other suitable frequency, and the time difference between sequential temperature measurements may vary over time, or it may be uniform. Because temperature changes do not tend to occur rapidly in a natural environment, and to conserve energy (e.g., the energy harvested via a non-battery energy source), the temperature sensing frequency may be set to a frequency of every few minutes or every few hours.

The output device(s) 112 is configured to provide an output based on the temperature data 114. This disclosure describes various types of output devices 112 that can be implemented with the device 102 and/or an object 104 including the device 102. In some examples, the output device(s) 112 may include a display(s), a light emitting element(s) (e.g., a light emitting diode(s) (LED(s))), an electrochromic material(s), a speaker(s), a haptic actuator(s), and/or a transmitter(s) (e.g., a transceiver, such as a wireless radio, antenna, or the like), as described herein. In some examples, providing the output based on the temperature data 114 includes sending the temperature data 114 (or a result determined by analyzing the temperature data 114) to an external device 118 and/or a remote system 120. FIG. 1 illustrates several example types of external devices 118, including a case 118(1) (e.g., a wall cabinet) for storing an external defibrillator, an external defibrillator 118(2), and/or a server(s) 118(P), although it is to be appreciated that other types of external devices 118 are contemplated, such as a personal computer (PC), a laptop computer, a tablet, a wearable device (e.g., a headset, glasses, a watch, etc.), or the like.

The device 102 and the external device 118 may be communicatively coupled. In some examples, this communicative coupling may be a short range communications connection 122. The communications connection 122 may be wired or wireless. For example, the electrodes 104(1) may be communicatively coupled to the external defibrillator 118(2) via a wire or a cable, and/or wirelessly (e.g., via WiFi, Bluetooth, etc.). Additionally, or alternatively, FIG. 1 illustrates that the device 102, the external device 118, and/or the remote system 120 may be communicatively coupled to one another via a communication network(s) 124, such as the Internet, a cellular network, a satellite network, or the like. Accordingly, the communication network(s) 124 may represent a wide-area network (WAN), in some examples, and, as such, the remote system 120 may be located at a geographically disparate location with respect to a location of the device 102 and/or the external device 118. In some examples, the external device 118 is also geographically remote from the device 102.

It is to be appreciated that the temperature data 114 (and/or a result determined by analyzing the temperature data 114) may be sent from the device 102 to the external device 118 directly (e.g., via the short range communications connection 122, or via the communication network(s) 124), from the device 102 to the remote system 120 directly (e.g., via the communication network(s) 124), and/or from the external device 118 to the remote system 120 directly (e.g., via the communication network(s) 124). As such, any of the devices/systems 102, 118, and/or 120 may process the temperature data 114 and/or forward results determined by analyzing the temperature data 114 to other devices.

FIG. 2 illustrates example components of a passive temperature-logging device 102. As mentioned above, at least some of the components shown as being part of the device 102 may represent components of the object 104 to which the device 102 is coupled. For example, if the object 104 is an external defibrillator 104(2), the external defibrillator 104(2) may have at least some of the components shown in FIG. 2, such as a processor(s) 200, memory(ies) 110, and/or an output device(s) 112. As such, a device 102 coupled to (e.g., built into, integrated with, etc.) the external defibrillator 104(2) may include components that the external defibrillator 104(2) does not already have. In some cases, this might be a temperature sensor(s) 108. In any case, FIG. 2 illustrates that the device 102 may include the temperature sensor(s) 108, the memory(ies) 110, and/or the output device(s) 112 introduced in FIG. 1. It is to be appreciated that individual components depicted in FIG. 2 may be hardware components, firmware components, and/or software components.

In some examples, the device 102 includes a processor(s) 200. In some examples, the processor(s) 200 includes a central processing unit (CPU), a graphics processing unit (GPU), both CPU and GPU, or another processing unit or component known in the art. The processor(s) 200 is operably connected to the memory 110. In various implementations, the memory 110 is volatile (such as random access memory (RAM)), non-volatile (such as read only memory (ROM), flash memory, etc.) or some combination of the two. The memory 110 stores instructions that, when executed by the processor(s) 200, cause the processor(s) 200 to perform various operations described herein. In various examples, the memory 110 stores methods, threads, processes, applications, objects, modules, any other sort of executable instruction, or a combination thereof. Examples depicted in FIG. 2 include a temperature sensing manager 202, an energy monitor 204, and a temperature analyzer 206. In some cases, the memory 110 stores files, databases, or a combination thereof. FIG. 2 depicts the memory 110 as storing temperature data 114 and potentially other data 208 (e.g., other environmental data) that may be used to implement the techniques and functionalities described herein. In some examples, the memory 110 includes RAM, ROM, electrically erasable programmable read-only memory (EEPROM), flash memory, or any other memory technology. In some examples, the memory 110 includes CD-ROMs, digital versatile discs (DVDs), content-addressable memory (CAM), and/or other optical storage, magnetic cassettes, magnetic tape, magnetic disk storage and/or other magnetic storage devices, and/or any other medium (e.g., non-transitory computer-readable medium) which can be used to store the desired information and which can be accessed by the processor(s) 200 and/or the device 102. Although a processor 200, a memory 110, and other components are depicted in FIG. 2 as being part of the device 102, it is to be appreciated that the device 102 may be implemented as an application-specific integrated circuit (ASIC), in some examples, with functionality similar to the functionality described with reference to one or more of the components shown in FIG. 2.

In some examples, the device 102 includes sensors 210. The sensors 210 may include the aforementioned temperature sensor(s) 108 configured to passively sense a temperature of an object 104 to which the device 102 is coupled. The temperature sensor(s) 108 can be implemented as any suitable type of temperature sensor, such as a thermistor, a thermocouple, a semiconductor-based integrated circuit (IC), or the like. Additionally, the sensors 210 may include a humidity sensor(s), an air pressure sensor(s) (or atmospheric pressure sensor(s)), an altitude sensor(s) (e.g., altimeter), an ambient light sensor(s), an accelerometer(s), an inertial measurement unit(s) (IMU(s)), a gyroscope(s), a gel integrity sensor(s), or the like. One or more of these types of sensors 210 may be utilized to determine various environmental conditions of the object 104 to which the device 102 is coupled, such as the humidity of the object's environment, the air pressure of the object's environment, the altitude of the object 104, the ambient light in the object's environment, motion of the object 104, or the like. The other data 208 stored in the memory 110 may represent a history of environmental parameter values sensed by one or more of these sensors 210, such as humidity values, air pressure values, altitude values, light values, motion/movement values, or the like, which may be sensed over time as a series of values, much like the temperatures (or temperature values) described in detail herein. Furthermore, the data 208 relating to these environmental parameters may be processed and/or used in a similar manner to the use of the temperature data 114 described herein. Accordingly, references to temperature data 114 throughout this disclosure may be interchanged with data relating to other environmental parameters, such as those described above.

In some examples, the device 102 includes an energy harvesting component 212 configured to harvest energy from a non-battery (or ambient) energy source. Such energy harvesting may occur at least when the object 104 does not have a power source, and/or when a mains electricity and/or a battery is inaccessible to the object 104, such as when a set of electrodes 104(1) is separated from an external defibrillator. Additionally, or alternatively, for battery-powered objects 104, such as an external defibrillator 104(2), such energy harvesting may occur at least when the object 104 is separated from mains electricity (e.g., not plugged into a power outlet). The harvested energy may be utilized for passive temperature sensing and/or passive temperature logging. For example, the harvested energy may be used to power the temperature sensor 108 for sensing the temperature of the object 104 and/or to power the memory 110 for storing the temperature data 114 representing a history 116 of temperatures sensed by the temperature sensor 108, and/or to power other components of the device 102, such as the output device 112. As mentioned above, the temperature sensor 108, in some examples, may not require any energy to function as a passive temperature sensor, as is the case with a passive RFID temperature sensor that is configured to sense a temperature (e.g., a temperature change) by detecting a change in voltage across a diode. In this example, the device 102 may omit an energy harvesting component(s) 212, and/or the device 102 may utilize the harvested energy to power other components besides the temperature sensor 108, such as the memory 110 for passive temperature logging (e.g., recording a series of temperatures sensed by the temperature sensor 108 over time).

In some examples, the energy harvesting component 212 may be configured to harvest energy from EM radiation in a particular spectrum(s), such as the radio spectrum, and/or particular frequency bands within the radio spectrum. For example, the energy harvesting component 212 may utilize, or include, an antenna(s) to harvest (or capture) energy from radio transmissions in the vicinity of the device 102. Such radio transmissions may be converted into electrical energy that is usable to power one or more components of the device 102. The radio waves may have various properties, such as frequency, wavelength, amplitude, and/or other properties. In some examples, the antenna(s) may be configured (e.g., tuned) to receive radio transmissions having a specific property and/or within a specific range of a property, such as a particular frequency range. In some examples, the radio transmissions that are the basis of the energy harvesting can media broadcast signals, cellular phone transmissions, wireless internet signals and/or other sources of radio energy radiation. In a heavily trafficked area, for example, there may be enough harvestable energy from cell phone use to power one or more components of the device 102 for passive temperature sensing and/or logging. In some examples, an antenna(s) used for harvesting energy from radio transmissions can harvest power in a range of about hundreds of microwatts to several milliwatts, or greater, of power, depending on the breadth of the frequency band(s) used for energy harvesting and/or the prevalence of radio transmissions in the vicinity of the device 102.

In some examples, radio transmissions are purposefully generated by an external device(s) 118 in order to provide harvestable energy to the energy harvesting component 212. Accordingly, an external device(s) 118 can transmit (or emit) EM radiation at any suitable time while in the vicinity of the device 102 (e.g., within a threshold distance of the device 102 that is suitable for receiving radio transmissions from the external device(s) 118), and the energy harvesting component 212 may harvest the energy from the received radio transmissions.

In some examples, the energy harvesting component 212 may utilize, or include, multiple antennas, such as a first antenna to harvest energy from first radio transmissions in a first frequency range, a second antenna to harvest energy from second radio transmissions in a second frequency range, and so on. The energy captured by each antenna can be utilized to power specific components of the device 102. For example, the energy harvested from the first antenna may be used to power the temperature sensor 108, the energy harvested from the second antenna may be used to power the memory 110, and so on.

In some examples, the energy harvesting component 212 may be configured to harvest energy from light, such as sunlight and/or artificial light sources (e.g., overhead lighting). The light can be visible light and/or non-visible light (e.g., infrared (IR) light, ultraviolet (UV) light, etc.). In an example, the energy harvesting component 212 may utilize, or include, a photovoltaic cell(s) (e.g., solar cell(s), solar panel(s), etc.) to harvest (or capture) energy from light emissions (e.g., sunlight). Such light emissions may be converted into electrical energy that is usable to power one or more components of the device 102. In some examples, the photovoltaic cell(s) may be configured to produce power in a range from about 1 to 200 watts per square meter, depending on the light source (e.g., sunlight verses artificial light). In some examples, the photovoltaic cell(s) may be configured to produce power in a range from about 1 to 400 watts per hour. In some examples, the photovoltaic cell(s) may double as an ambient light sensor(s) of the device 102. That is, the device 102 may leverage the photovoltaic cell(s) for sensing ambient light so that a separate light sensor is not needed.

In some examples, the energy harvesting component 212 may be configured to harvest energy from the motion of a mass. For example, the energy harvesting component 212 may utilize, or include, a mechanical motion device(s) to harvest (or capture) energy from the motion of a mass that is in or on the device 102. The kinetic energy of the mass may be converted into electrical energy that is usable to power one or more components of the device 102. Examples of such mechanical motion devices include micro-electromechanical systems (MEMS), a magnet that is configured to move within a coil of wire, a rotating weight/pendulum, etc. Such devices may include a generator (e.g., a generator that includes a capacitor charged by mechanical motion of a mass). Accordingly, the generator may be configured to convert kinetic energy into electrical energy.

In some examples, the energy harvesting component 212 may be configured to harvest thermal energy. For example, the energy harvesting component 212 may utilize, or include, a thermocouple(s) to harvest (or capture) energy based on a temperature differential, such as a temperature differential based on temperature fluctuations in the environment of the device 102. The thermal energy may be converted into electrical energy that is usable to power one or more components of the device 102. In an illustrative example, a set of electrodes 104(1) may have a first portion/component that changes temperature more quickly than a second portion/component of the electrodes 104(1). Accordingly, a thermocouple of the device 102 may be configured to generate electrical energy using the temperature differential between the different portions/components of the electrodes 104(1). As another example, the object 104 may represent a human (e.g., a patient), the device 102 may be worn by the human, and the temperature differential between the body temperature of the human and the environment of the human may be used to generate electrical energy.

In some examples, the object 104 may be coupled to an external device(s) 118, and the external device 118 is configured to use a certain amount of power (e.g., battery power) to wake itself up periodically. In this example, the energy harvesting component 212 may be configured to divert a portion of that power and use the diverted power to power one or more components of the device 102. In this example, the device 102 may be configured to harvest energy from a battery. For example, the device 102 may be coupled to a set of electrodes 104(1), and a device 102 coupled to the electrodes 104(1) may “harvest” a portion of the energy used by an external defibrillator 118(2) to periodically wake itself up.

In some examples, the device 102 includes an energy storage component(s) 214 configured to store energy (e.g., the energy harvested by the energy harvesting component 212) as stored energy. In some examples, the energy storage component(s) 214 represents at least one of a battery(ies) (e.g., a small, inexpensive battery, such as a 1.5 volt coin battery or similar, loaded with enough energy to power one or more components of the device 102 for purposes of temperature sensing and/or logging), a capacitor, or some other component configured to store energy. If the object 104 is an external defibrillator 104(2), for instance, such an energy storage component(s) 214 may be separate from a primary power source (e.g., a primary battery) used by the external defibrillator 104(2) for its core function(s) of administering defibrillation therapy to patients. In this manner, sensing and/or logging the temperature of the object 104 (in this case, the external defibrillator 104(2)) would not deplete the primary power source (e.g., the primary battery) of the object 104. Even if the energy storage component(s) 214 runs out of stored energy before the expiration of the object 104, it could provide enough power to log the temperature of the object 104 for at least a portion of the shelf life of the object 104, which may be useful despite not knowing the temperature of the object 104 for a remaining portion of its shelf life. It is to be appreciated that the energy storage component(s) 214 may be used in conjunction with, or in lieu of, the energy harvesting component(s) 212. That is, in some examples, the device 102 may not include an energy harvesting component(s) 212, but may include an energy storage component(s) 214 (e.g., a battery(ies)) dedicated for temperature logging for as long as the energy storage component(s) 214 has stored energy to use for such temperature sensing and/or logging. In this manner, the dedicated energy storage component(s) 214 can be used for temperature logging instead of using a primary power source (e.g., a primary battery) of the object 104 to do so.

In some examples, the energy storage component(s) 214 is configured to provide an energy buffer (e.g., an amount of stored energy) that is usable to power one or more components of the device 102 when energy cannot be harvested, or when only a small amount of energy can be harvested, by the energy harvesting component(s) 212. For example, in an implementation where the energy harvesting component 212 utilizes, or includes, a photovoltaic cell(s), stored energy maintained by the energy storage component 214 may be used for temperature sensing and/or logging during nighttime hours when no sunlight is available, and/or when no artificial light sources are available for harvesting energy therefrom. In another example, the energy harvesting component 212 utilizes, or includes, an antenna(s), and stored energy maintained by the energy storage component 214 may be used for temperature sensing and/or logging when there are little-to-no radio transmissions in the vicinity of the device 102 (e.g., at night, when an area is not as heavily trafficked with cell phone users as compared to an amount of cell phone users trafficking the area during the day). Accordingly, the energy storage component(s) 214 can provide energy reserves to allow passive temperature sensing and/or logging during periods of low energy harvesting. In some examples, the energy storage component 214 may be pre-charged with an amount of energy at a time of manufacturing the device 102 and/or at a time of manufacturing the object 104 that includes the device 102. In this manner, passive temperature sensing and/or logging can be initiated using the initially-stored energy as an energy buffer while waiting for enough energy to be harvested via the energy harvesting component 212. In some examples, the harvested energy and/or the stored energy stored by the energy storage component(s) 214 may be used to power other low-current circuits of the device 102, such as a main battery failure indicator, which may be provided via the output device(s) 112.

In some examples, the output device(s) 112 may include a display(s) 216, a light emitting element(s) 218 (e.g., a LED(s)), an electrochromic material(s) 220, a speaker(s) 222, a haptic actuator(s) 224, and/or a transceiver(s) 226 (e.g., a wireless radio, antenna, or the like). As noted above, the output device(s) 112 is/are configured to provide an output(s) based on the temperature data 114. In an example, the processor(s) 200 is configured to execute the temperature analyzer 206 (e.g., computer-executable instructions) to analyze the temperature data 114 representing the temperature history 116 of the object 104, and the display(s) 216 is configured to output an indication (e.g., a visual indication, such as a color, text, graphics, animations, video, etc.) of a usability of the object 104 using a result of the analyzing the temperature data 114. For example, the processor(s) 200 may execute instructions that cause the display 216 to output a first color (e.g., red) indicating that the object 104 should be replaced, and/or a second color (e.g., green) indicative of the object 104 being usable. As another example, the display 216 is configured to output text (e.g., text indicative of whether the object 104 has any life remaining, an amount of remaining life of the object 104, a notification or a warning that the object 104 should be replaced and/or that the object 104 is about to expire, etc.). For instance, the processor(s) 200 may be configured to execute the temperature analyzer 206 (e.g., computer-executable instructions) to predict a time remaining until the object 104 is expired using a result of analyzing the temperature history 116, and the processor(s) 200 may execute instructions that cause the display 216 to output an indication of the time remaining. In some examples, the processor(s) 200 may be configured to output a notification or a warning about a remaining life of the object if the object 104 is within a threshold time from expiration; otherwise, such a notification or warning may not be output until that threshold is reached. A user may be able to access such information on request.

In some examples, the light emitting element(s) 218 is configured to output a usability indication of the object 104 in the form of a visual indication, such as a color of light, a frequency of light pulses (e.g., blink frequency), an intensity of light (e.g., bright light, dim light, etc.), or the like. For example, a red light and/or a flashing light is an example of a usability indication indicating that the object 104 should be replaced.

In some examples, the electrochromic material(s) 220 is configured to output a usability indication of the object 104 in the form of a color. Electrochromic materials, also known as chromophores, affect the optical color or opacity of a surface when a voltage is applied. Examples of electrochromic materials 220 include metal oxides, tungsten oxide, molybdenum, titanium oxide, niobium oxides, or polypyrrole.

In some examples, the speaker(s) 222 is configured to output a usability indication of the object 104 in the form of an audio indication, such as a voice prompt, a sound, or the like. In some examples, the haptic actuator(s) 224 is configured to output a usability indication of the object 104 in the form of a haptic indication, such as a vibration, which may be audible as well.

In some examples, the transceiver(s) 226 is configured to configured to send the temperature data 114 (or a result determined by analyzing the temperature data 114, such as an amount of time that a temperature of the object 104 was outside of a predetermined temperature range) to an external device(s) 118 and/or to a remote system 120. In some examples, the external device(s) 118 and/or the remote system 120 may be configured to further process (e.g., analyze) the temperature data 114 (or the result) received from the device 102. A push model or a pull model may be implemented. In a push model, temperature data 114 (or a result determined therefrom) may be sent via the transceiver 226 periodically and/or in response to an event determined by the device 102. For instance, the processor(s) 200 may be configured to execute the temperature analyzer 206 (e.g., computer-executable instructions) to analyze the temperature data 114 to determine whether one or more of the temperatures in the history 116 of temperatures satisfies a threshold, and, if so, the transceiver 226 may send the temperature data 114 (or a result determined therefrom) to the external device(s) 118 and/or the remote system 120 in response to the temperature(s) satisfying the threshold. In a pull model, the processor(s) 200 may execute computer-executable instructions to cause the transceiver 226 to send the temperature data 114 (or a result determined therefrom) in response to receiving a request (e.g., an interrogation signal) from an external device(s) 118 and/or a remote system 120. For example, the external device(s) 118 may interrogate (e.g., periodically and/or in response to an event) the device 102 for updated temperature data 114, and the device 102 may send, via the transceiver 226, in response to the interrogation signal, updated temperature data 114 representing the temperature history 116 of the object 104. Such an interrogation signal may be a radio frequency signal. In some examples, the transceiver 226 is configured to transmit a usability indication of the object 104 to an external device(s) 118 for display of the usability indication on a display of the external device(s) 118 and/or for output of the usability indication via a speaker of the external device(s) 118. In this example, the external device 118 may be a medical device (e.g., an external defibrillator 118(2), a mobile phone, a tablet, or the like.

In some examples, the processor(s) 200 is configured to execute the energy monitor 204 (e.g., computer-executable instructions) to determine an amount of energy stored by the energy storage component(s) 214. By analyzing the amount of the stored energy, various actions can be taken. For example, the processor(s) 200 may be configured to execute the temperature sensing manager 202 (e.g., computer-executable instructions) to adjust a frequency at which the temperature sensor 108 periodically senses the temperature of the object 104 based on (e.g., by analyzing) the amount of energy stored by the energy storage component 214. In this manner, if energy reserves are relatively low, the temperature-sensing frequency may be decreased to conserve energy, and if energy reserves are relatively high, the temperature-sensing frequency may be increased, seeing as how the device 102 is not as concerned with running out of energy reserves.

In some examples, the temperature-sensing frequency (i.e., the frequency at which the temperature sensor 108 is configured to periodically sense the temperature of the object 104) is based at least in part on a thermal mass of the object 104. That is, if the object 104 changes temperature relatively slowly, the temperature sensing frequency can be set (e.g., by the processor 200 executing the temperature sensing manager 202) to a relatively low frequency. On the other hand, if the object 104 changes temperature relatively quickly, the temperature sensing frequency can be set to a relatively high frequency. In some examples, the temperature-sensing frequency can be set to a frequency within a range of about once per five minutes to once per day. That is, the temperature logging may occur at least daily, or multiple times a day. However, to conserve energy, and based on the thermal mass of the object, the temperature-sensing frequency may be set to (e.g., adjustable to) a frequency of about once every five minutes, in some examples. In some examples, the temperature-sensing frequency is aligned with a transmission frequency (e.g., a frequency at which the device 102 transmits temperature data 114 (or a result determined therefrom) to an external device), and/or an interrogation frequency (a frequency at which an external device 118 periodically sends interrogation signals to the device 102 to receive updated temperature data 114 representing the temperature history 116 of the object 104).

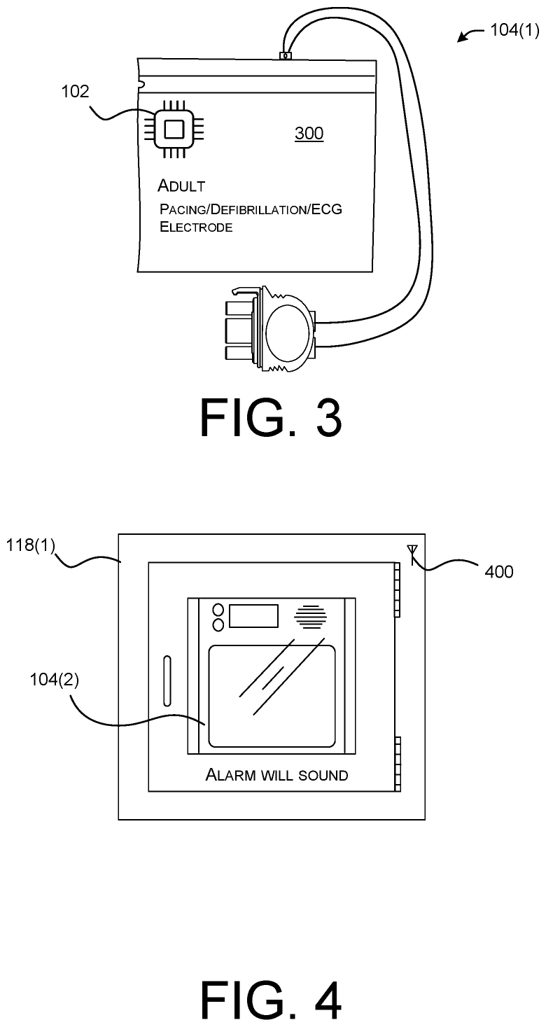

FIG. 3 illustrates an example passive temperature-logging device 102 coupled to a packaged set of electrodes 104(1), the passive temperature-logging device 102 configured to passively log the temperature of the packaged electrodes 104(1), according to the techniques described herein. Having the device 102 coupled to the packaged electrodes 104(1) allows for obtaining a record of the temperature history of the electrodes 104(1) while keeping the electrodes 104(1) packaged (e.g., within a hermetically-sealed container 300 (e.g., pouch, reusable container, etc.)) to maintain a level of humidity inside of the sealed container 300, thereby maximizing the shelf life of the electrodes 104(1) while passively logging the temperature of the electrodes 104(1). Knowledge of the temperature history of the electrodes 104(1) allows an end user to avoid prematurely discarding “perfectly good” electrodes 104(1). Alternatively, a user may discard electrodes 104(1) at an appropriate time if, say, the electrodes 104(1) have been improperly stored (e.g., at temperatures that are too hot).

In FIG. 3, the electrodes 104(1) are stored in the container 300 separate from an external defibrillator 118(2) with which the electrodes 104(1) are configured to be used. In this example, the energy harvesting component 212 may be configured to harvest energy from a non-battery energy source when the electrodes 104(1) are stored in the container 300. In some examples, the container 300 is reusable. In other examples, the container 300 is single use, such as a foil pouch. In some examples, the device 102, or at least one or more components thereof, are disposed on an external surface of the container 300. For example, the energy harvesting component 212 (e.g., antenna(s), photovoltaic cell(s), etc.) and/or the transceiver 226 of the device 102 may be external to the container 300 to avoid blockage of EM radiation by the container 300. In some examples, at least some of the components of the device 102 may be inside of the container 300. For instance, a mechanical motion device(s) configured to harvest (or capture) energy from the motion of a mass may be disposed inside of the container 300. Accordingly, the device 102 can be inside, outside, or partially inside and partially outside of the container 300.

FIG. 4 illustrates an example external defibrillator 104(2) stored in a case 118(1) (e.g., a wall cabinet, such as those in airports and other public venues), the case 118(1) including a component 400 that is configured to interrogate a passive temperature-logging device 102 coupled to an external defibrillator 104(2) that is stored within the case 118(1), according to the techniques described herein. For example, the temperature sensor 108 of the device 102 may be configured to sense the temperature of the external defibrillator 104(2) in response to an interrogation signal received from the case 118(1) (e.g., an interrogation signal received from the component 400 of the case 118(1)). Additionally, or alternatively, temperature data 114 (or a result determined therefrom) may be sent to the component 400 of the case 118(1) in response to the interrogation signal, and the component 400 may relay the temperature data 114 (or the result) to another computing system, such as a remote system 120. In some examples, the component 400 is a transceiver that is configured to send interrogation signals periodically and/or in response to an event, and to receive data from the device 102.

In some examples, the case 118(1) may store an external defibrillator 118(2) and a set of electrodes 104(1) configured to be used with the external defibrillator 118(2). In this example, the device 102 may be coupled to the set of electrodes 104(1) within the case 118(1), and the temperature sensor 108 of the device 102 may be configured to sense the temperature of the electrodes 104(1) in response to an interrogation signal received from the case 118(1) (e.g., an interrogation signal received from the component 400 of the case 118(1)) and/or in response to an interrogation signal received from the external defibrillator 118(2). In the examples described with reference to FIG. 4, the primary battery of the external defibrillator 104(2), 118(2) that is stored within the case 118(1) is not depleted in order to passively log the temperature of external defibrillator 104(2) and/or the electrodes 104(1) stored within the case 118(1). Accordingly, the external defibrillator 104(2), 118(2) can remain ready for performing its core functionality of administering defibrillation therapy to a person in need of assistance.

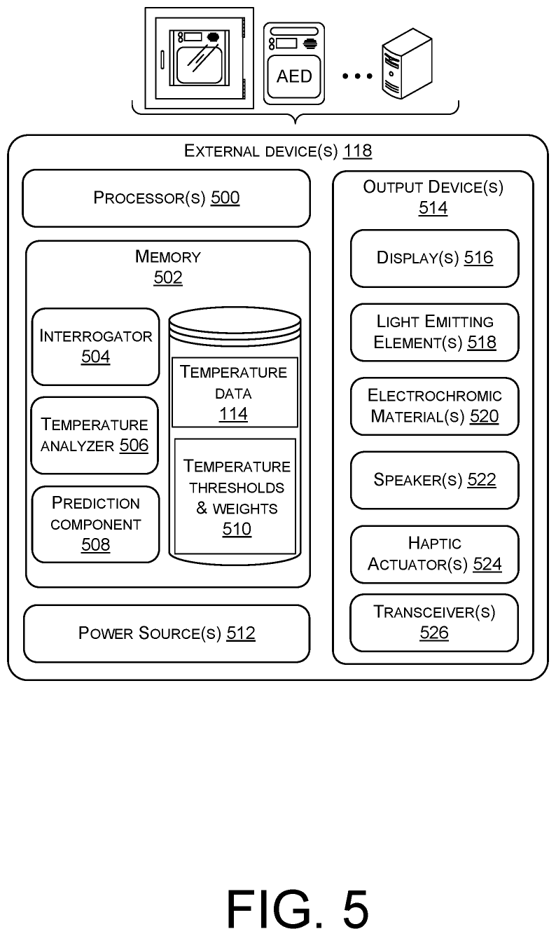

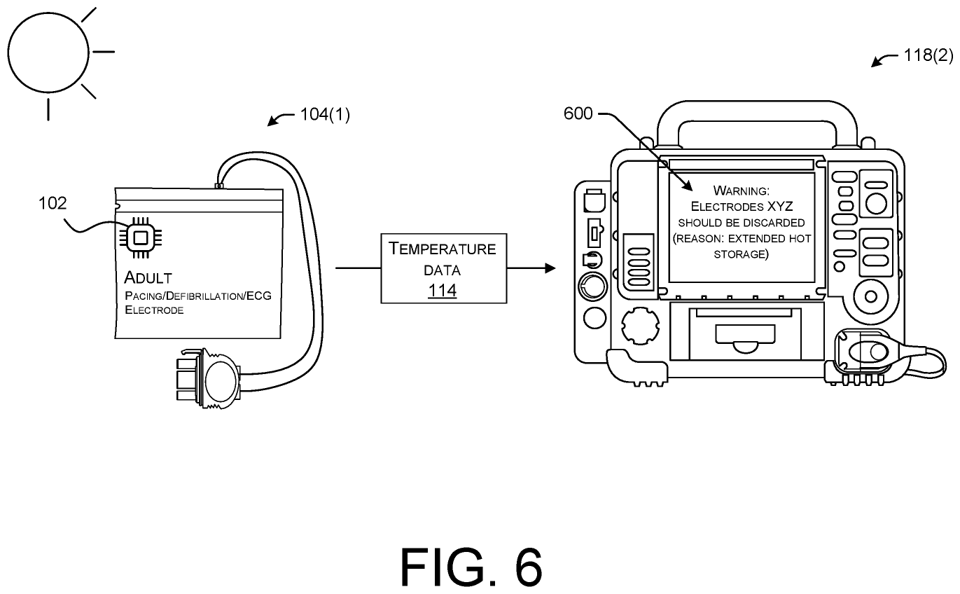

FIG. 5 illustrates example components of an external device 118 configured to receive, from a passive temperature-logging device 102, temperature data 114 representing a temperature history 116 of an object 104 to which the passive temperature-logging device 102 is coupled, according to the techniques described herein. It is to be appreciated that individual components depicted in FIG. 5 may be hardware components, firmware components, and/or software components. In some examples, the external device 118 includes a processor(s) 500, which may be similar to the processor(s) 200 described above with reference to FIG. 2. The processor(s) 500 is operably connected to a memory 502, which may be similar to the memory 110 described above with reference to FIGS. 1 and 2. In various examples, the memory 502 stores methods, threads, processes, applications, objects, modules, any other sort of executable instruction, or a combination thereof. Examples depicted in FIG. 5 include an interrogator 504, a temperature analyzer 506, and a prediction component 508. In some cases, the memory 502 stores files, databases, or a combination thereof. FIG. 5 depicts the memory 502 as storing temperature data 114, as well as temperature thresholds and corresponding weights 510, which may be used to implement the techniques and functionalities described herein.

In some examples, the external device 118 includes a power source(s) 512. The power source(s) 512 may include a battery(ies), such as a primary battery that is used to carry out core functions of the external device 118 when the external device 118 is separated from mains electricity. It is to be appreciated that the external device 118 may be configured to be coupled to mains electricity, in some examples, in order to receive power from the grid (e.g., the external device 118 can be plugged into a power outlet). This can be in combination with a battery power source 512.

In some examples, the external device 118 includes one or more output devices 514, which may be similar to the output device(s) 112 described with reference to FIGS. 1 and 2. For example, the output device(s) 514 may include a display(s) 516, a light emitting element(s) 518 (e.g., a LED(s)), an electrochromic material(s) 520, a speaker(s) 522, a haptic actuator(s) 524, and/or a transceiver(s) 526. These example types of output devices may be similar to the display(s) 216, the light emitting element(s) 218, the electrochromic material(s) 220, the speaker(s) 222, the haptic actuator(s) 224, and the transceiver(s) 226, respectively, as described above with reference to FIG. 2. In some examples, the output device(s) 514 is/are configured to provide an output(s) based on the temperature data 114 received from the device 102. In an example, the processor(s) 500 is configured to execute the temperature analyzer 506 (e.g., computer-executable instructions) to analyze the temperature data 114 representing the temperature history 116 of an object 104, and the display(s) 516 is configured to output an indication (e.g., a visual indication, such as a color, text, graphics, animations, video, etc.) of a usability of the object 104 using a result of the analyzing the temperature data 114. For example, the processor(s) 500 may execute instructions that cause the display 516 to output a first color (e.g., red) indicating that the object 104 should be replaced, and/or a second color (e.g., green) indicative of the object 104 being usable. As another example, the display 516 is configured to output text (e.g., a remaining life of the object 104, a notification or a warning that the object 104 should be replaced, etc.). In some examples, the processor(s) 500 may be configured to output a notification or a warning about a remaining life of the object if the object 104 is within a threshold time from expiration; otherwise, such a notification or warning may not be output until that threshold is reached. A user may be able to access such information on request. In some examples, the processor(s) 500 may be configured to execute the prediction component 508 (e.g., computer-executable instructions) to predict a time remaining until the object 104 is expired using a result of analyzing the temperature history 116, and the processor(s) 500 may execute instructions that cause the display 516 to output an indication of the time remaining.

In some examples, the prediction component 508 and/or the temperature analyzer 506 may use the multiple different temperature thresholds and corresponding weights 510 to analyze the temperature history 116 of an object. For example, the multiple different temperature thresholds and corresponding weights 510 may include a first threshold associated with a first weight, a second threshold associated with a second weight, and so on. Analyzing the temperature history, in some examples, may include determining whether a subset of sequential temperatures in the temperature history 116 (or a statistic computed therefrom, such as an average temperature) exceed a single threshold or multiple thresholds, determining an integral associated with the subset of sequential temperatures that exceed the threshold(s) (e.g., compute the integrated thermal energy over time), and determining a value using the integral and a weight corresponding to the highest threshold that is exceeded by the sequential temperatures. For example, if the first threshold is less than the second threshold, and if a subset of sequential temperatures exceed the first threshold but do not exceed the second threshold, the value may be determined using the integral and the first weight associated with the first threshold. On the other hand, if the subset of sequential temperatures exceeds both the first threshold and the second threshold, the value may be determined using the integral and the second weight. In any case, the value may be used to predict the time remaining until the object 104 is expired. In some examples, the value is input into a model (e.g., a mathematical model, a machine-learning model, etc.) to estimate or predict the remaining life of the object 104.

In some examples, the multiple temperature thresholds and weights 510 may include progressively increasing temperature thresholds with corresponding weights that progressively increase in step with the progressively increasing temperature thresholds. In these examples, the amount of time that the temperature of an object 104 exceeds the highest threshold may cause the prediction of remaining life of the object to decrease most quickly, as compared to temperatures that exceed any of the lower thresholds but not the highest threshold. To illustrate, if the temperature history 116 of the object 104 indicates that the object 104 was between 20 and 30 degrees Celsius (C) over a time period of five years, the remaining life of the object 104 may be predicted to be longer than if the temperature history 116 indicated that the object 104 exceeded 200 degrees for a period of time. In some examples, a formula used to predict the remaining life of the object 104 may involve subtracting life if the temperature of the object 104 exceeded a threshold temperature at any point, and adding life if the temperature was below the same threshold or a different threshold. This may be because freezing an object 104 actually extends the shelf life of the object 104. This is not to say that the object 104 is functional at such temperatures. Accordingly, a usability indication (e.g., determining the readiness of the object 104) may be a separate consideration from a prediction of the remaining life of the object 104. For example, the remote system 120 (e.g., a fleet monitoring system) may determine, from the temperature history 116 of an object 104, that the object 104 (e.g., an external defibrillator 104(2) with serial number XYZ) spends half of its time at temperatures that it can't be expected to function (e.g., below a certain temperature, such as an external defibrillator 104(2) used by ski patrol). That is, the remote system 120 may be configured to determine if the external defibrillator 104(2), in the above example, is able to defibrillate a patient based on the temperature history 116, and a temperature history 116 that exhibits temperatures below a threshold temperature may indicate that such an object 104 is not ready to perform its intended function(s).

In some examples, the light emitting element(s) 518, the electrochromic material(s) 520, the speaker(s) 522, and/or the haptic actuator(s) 524 is/are configured to output a usability indication of the object 104, as described above with respect to the similar types of output devices 112 described with reference to FIG. 2.

In some examples, the transceiver(s) 526 is configured to configured to receive the temperature data 114 (or a result determined by analyzing the temperature data 114, such as an amount of time that a temperature of the object 104 was outside of a predetermined temperature range) from the device 102. In some examples, the external device 118 may be configured to further process (e.g., analyze) the temperature data 114 (or the result) received from the device 102, and/or to forward the temperature data 114 (or a result determined therefrom) to a remote system 120. Again, a push model or a pull model may be implemented. In a push model, the external device 118 may receive (periodically and/or in response to an event) temperature data 114 (or a result determined therefrom) from the device 102. In a pull model, the processor(s) 500 may execute the interrogator 504 (e.g., computer-executable instructions) to cause the external device 118 to send to the device 102, via the transceiver 526, an interrogation signal(s) to receive updated temperature data 114 representing the temperature history 116 of an object 104 to which the device 102 is coupled. The interrogation signals may be sent to the device 102, via the transceiver 526, periodically and/or in response to an event. Such interrogation signals may be radio frequency signals.

In some examples, the processor(s) 500 is configured to execute the temperature analyzer 506 (e.g., computer-executable instructions) to determine, by analyzing the temperature history 116 of an object 104, an amount of time that a temperature of the object 104 was outside of a predetermined temperature range, and the transceiver 526 is configured to send the amount of time to a remote system 120. In this example, the remote system 120 may represent an asset management system configured to manage a fleet of objects, and the object 104 whose temperature history 116 is analyzed may be one of the objects in the fleet.

FIG. 6 illustrates an example external defibrillator 118(2) that is configured to receive temperature data 114 from a passive temperature-logging device 102 coupled to a set of electrodes 104(1) and to output an indication 600 of a usability of the electrodes 104(1) based on the received temperature data 114, according to the techniques described herein. To illustrate, the temperature sensor 108 of the device 102 may passively sense the temperature of the electrodes 104(1) over time, such as by using energy harvested from a non-battery energy source, by detecting a change in voltage across a diode, or the like, as described herein. Temperature data 114 representing a history of the temperatures sensed by the temperature sensor 108 may be stored in the memory 110 to log the temperatures, and the device 102 may send, via the transceiver 226, the temperature data 114 to the external defibrillator 118(2). The processor(s) 500 of the external defibrillator 118(2) may execute the temperature analyzer 506 (e.g., computer-executable instructions) to analyze the temperature data 114 (or the temperature history 116 represented by the temperature data 114), and, using a result of the analysis, the processor(s) 500 may cause an indication 600 of a usability of the electrodes 104(1) to be output via an output device(s) 514 of the external defibrillator 118(2). In the example of FIG. 6, the usability indication 600 is output via the display(s) 516 as text that reads “Warning: Electrodes XYZ should be discarded (reason: extended hot storage).” This is merely an example of a usability indication 600 that indicates a usability of the electrodes 104(1), and other types of usability indications 600 are contemplated, as described herein.

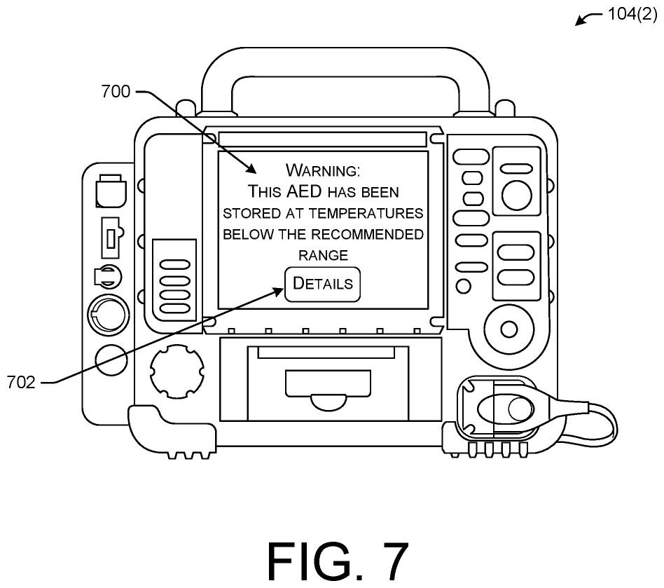

FIG. 7 illustrates an example external defibrillator 104(2) configured to output an indication 700 of a usability of the external defibrillator 104(2) based on a temperature history 116 of the external defibrillator 104(2) obtained by a passive temperature-logging device 102 coupled to the external defibrillator 104(2), according to the techniques described herein. In the example of FIG. 7, the passive temperature-logging device 102 is not shown because it is contemplated that the device 102 may be internal to the housing of the external defibrillator 104(2), such as by being built into the external defibrillator 104(2) at a time of manufacture. To illustrate, the temperature sensor 108 of the device 102 may passively sense the temperature of the external defibrillator 104(2) over time, such as by using energy harvested from a non-battery energy source, by detecting a change in voltage across a diode, or the like, as described herein. Temperature data 114 representing a history of the temperatures sensed by the temperature sensor 108 may be stored in the memory 110 to log the temperatures, and the processor(s) 200 may execute the temperature analyzer 206 (e.g., computer-executable instructions) to analyze the temperature data 114 (or the temperature history 116 represented by the temperature data 114), and, using a result of the analysis, the processor(s) 200 may cause an indication 700 of a usability of the external defibrillator 104(2) to be output via an output device(s) 112. In the example of FIG. 7, the usability indication 700 is output via a display(s) 216 of the external defibrillator 104(2) as text that reads “Warning: This AED has been stored at temperatures below the recommended range.” This is merely an example of a usability indication 700 that indicates a usability of the external defibrillator 104(2), and other types of usability indications 700 are contemplated, as described herein. In some examples, a selectable element 702 may be displayed in conjunction with, or as part of, the usability indication 700, the selectable element 702, when selected, providing more details regarding the temperature history 116 and/or the usability of the external defibrillator 104(2). For example, a user may select the selectable element 702 (e.g., a Details button on the display 216) to learn more about the temperature history 116 of the external defibrillator 104(2) (e.g., to see a list, a plot, a graph, or the like, of temperatures logged and times at which the temperatures were logged, statistics, such as average temperature over a time period, etc.), and/or to learn more about the readiness of the external defibrillator 104(2) (e.g., to determine if the external defibrillator 104(2) has warmed up to a suitable operating temperature, etc.).

The processes described herein represent sequences of operations that can be implemented in hardware, software, or a combination thereof. In the context of software, the blocks represent computer-executable instructions stored on one or more computer-readable storage media that, when executed by a processor(s), perform the recited operations. Generally, computer-executable instructions include routines, programs, objects, components, data structures, and the like that perform particular functions or implement particular abstract data types. The order in which the operations are described is not intended to be construed as a limitation, and any number of the described operations can be combined in any order and/or in parallel to implement the processes. In some examples, an operation(s) of the process may be omitted entirely. Moreover, the processes described herein can be combined in whole or in part with each other or with other processes.

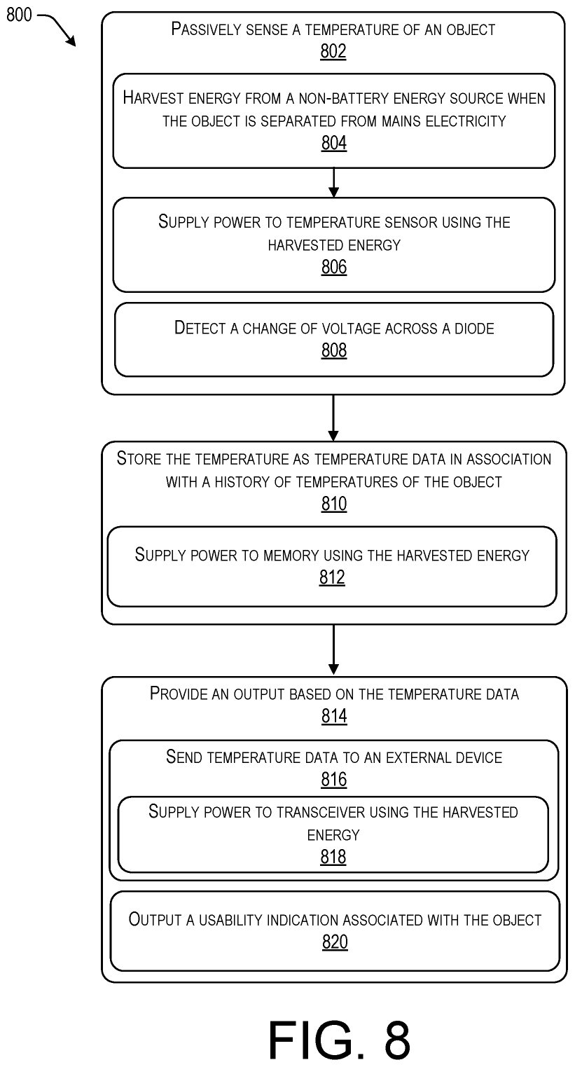

FIG. 8 illustrates an example process 800 for passively logging the temperature of an object 104 and providing an output based on the passively-logged temperature, according to the techniques described herein. The process 800 may be implemented by a device 102 coupled to an object 104, by an object 104 including the device 102, or by another suitable device or system. For discussion purposes, the process 800 is described with reference to the previous figures.

At 802, a temperature sensor 108 of a device 102 that is coupled to an object 104 may sense a temperature of the object 104. The object 104 may be any suitable type of object, as described herein, such as a medical device (e.g., a set of electrodes 104(1), an external defibrillator 104(2), etc.). In some examples, the temperature sensor 108 senses the temperature of the object 104 using energy harvested from a non-battery energy source, as indicated by sub-blocks 804 and 806.

At 804, the device 102 may harvest energy from a non-battery energy source. The non-battery energy source may include EM radiation, such as sunlight, artificial light, and/or EM radiation in a particular spectrum(s), such as the radio spectrum, and/or particular frequency bands within the radio spectrum. Additionally, or alternatively, the device 102 may harvest, at block 804, kinetic energy from the motion of a mass, thermal energy from a temperature differential, and/or any other suitable non-battery energy source described herein. In some examples, the energy is harvested at block 804 when the object 104 is separated from mains electricity and/or when the object 104 is separated from an external device with a power source. For example, if the object 104 is a medical device, such as an external defibrillator 104(2), the energy may be harvested at block 804 when the medical device (e.g., external defibrillator 104(2)) is separated from mains electricity. As another example, if the object 104 is a set of electrodes 104(1), the energy may be harvested at block 804 when the electrodes 104(1) are separated from an external defibrillator 118(2), for example. In examples where the object 104 is a set of electrodes 104(1), the electrodes 104(1) may be stored in a container 300 separate from an external defibrillator 118(2), and the energy harvesting component 212 of the device 102 coupled to the electrodes 104(1) may be configured to harvest, at block 804, the energy when the electrodes 104(1) are stored in the container 300.

At 806, power may be supplied (e.g., by the energy harvesting component 212) to the temperature sensor 108 using the energy harvested at block 804. In this manner, the temperature sensor 108 is configured to be powered by the harvested energy for passive temperature sensing, in some examples.

At 808, as an alternative approach to harvesting energy, the temperature sensor 108 may sense a temperature of the object 104 by detecting a change in voltage across a diode. For example, the temperature sensor 108 may represent a passive RFID temperature sensor, which may not require any energy to function as a passive temperature sensor.

At 810, the temperature may be stored in a memory 110 as temperature data 114. For example, the sensed temperature may be stored in association with a history 116 of temperatures of the object 104 that were previously sensed by the temperature sensor 108. In this manner, a temperature history 116 can be recorded in the memory 110 over time as temperatures are sensed. That is, the temperature data 114 stored in the memory 110 may represent a history 116 of temperatures sensed by the temperature sensor 108, wherein the temperature sensed at block 802 is one of the temperatures. The memory 110 may represent a memory of the device 102 or a memory of the object 104 including the device 102, as described herein.

At 812, power may be supplied (e.g., by the energy harvesting component 212) to the memory 110 using the energy harvested at block 804. In this manner, the memory 110 is configured to be powered by the harvested energy for passive temperature logging, in some examples.

At 814, an output may be provided via an output device 112 based on the temperature data 114. The output device(s) 112 may represent an output device of the device 102 or an output device of the object 104 including the device 102, as described herein. The output device(s) 112 can be any suitable type of output device described herein, such as a display 216, a light emitting element 218, a speaker 222, a transceiver 226, or the like.

At 816, as one example, the output device 212 includes a transceiver 226, and providing the output includes sending the temperature data 114 to an external device 118 external to the object 104. In some examples, the external device 118 to which the temperature data 114 is sent is a server 118(P) remote from the object 104. In some examples, in addition to, or in lieu of, sending the temperature data 114, a result determined by analyzing the temperature data 114 may be sent to the external device 118 (e.g., the server 118(P)) at block 816. In some examples, the server 118(P may further process the temperature data 114 and/or the result determined therefrom, and may report information to a remote system 120 (e.g., an asset management system, such as a fleet manager). The information reported may include a percentage of time that the object 104 was stored outside of a predetermined temperature range. This could be used for the purpose of helping the owner/manager of a fleet of objects 104 identify inappropriate storage situations so that the storage situations can be changed. The information reported may additionally, or alternatively, include a percentage of time that the object 104 is not ready because of storage temperature. As another example, the object 104 may represent a patient in a hospital) whose temperature is passively logged by the device 102 coupled to the patient, and the external device 118 that receives the temperature data 114 (or a result determined therefrom) may be a user device (e.g., a phone, tablet, etc.) that outputs related information that helps a doctor or a nurse make a more informed decision about how to care for the patient.

At 818, power may be supplied (e.g., by the energy harvesting component 212) to the transceiver 226 using the energy harvested at block 804. In this manner, the transceiver 226 is configured to be powered by the harvested energy for passive temperature transmission, in some examples.

At 820, providing the output may include outputting an indication of a usability (or a readiness) of the object 104 by analyzing the temperature data 114. In some examples, the usability indication may be output at block 820 as a warning or a notification that the object 104 should be discarded. In some examples, the usability indication may be output at block 820 as an indication of a time remaining until the object 104 is expired, an indication that a temperature of the object 104 was outside of a predetermined temperature range (or an indication of an amount of time that the temperature of the object 104 was outside of the range), or the like.

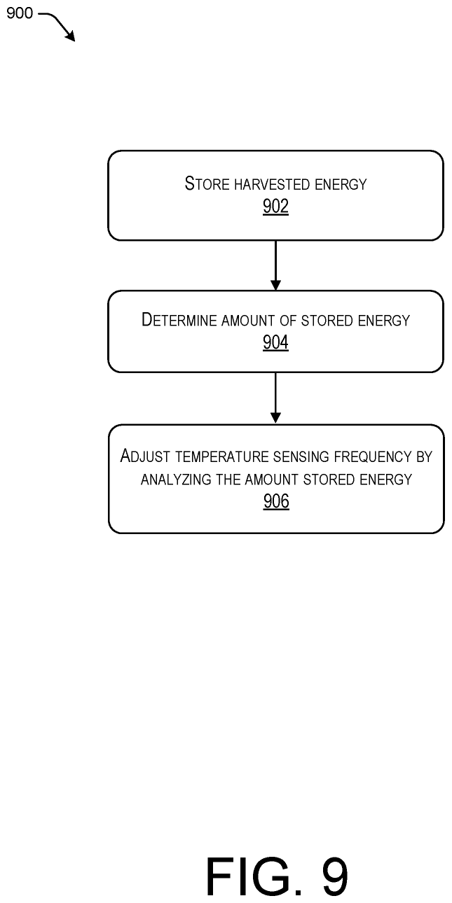

FIG. 9 illustrates an example process 900 for adjusting a temperature-sensing frequency of a passive temperature-logging device 102 based on an amount of harvested energy that is currently stored by the passive temperature-logging device 102, according to the techniques described herein. The process 900 may be implemented by a device 102 coupled to an object 104, by an object 104 including the device 102, or by another suitable device or system. For discussion purposes, the process 900 is described with reference to the previous figures.

At 902, an energy storage component 214 may store energy as stored energy. The energy stored at block 902 may have been harvested by the energy harvesting component 212, as described herein. As energy is harvested and as energy is used for passive temperature sensing and/or logging, as described herein, it can be appreciated that energy reserves (e.g., stored energy available to the device 102) may fluctuate. The energy storage component 214 may represent an energy storage component of the device 102 or an energy storage component of an object 104 including the device 102, as described herein. The object 104 may be any suitable type of object, as described herein, such as a medical device (e.g., a set of electrodes 104(1), an external defibrillator 104(2), etc.).

At 904, a processor 200 may determine an amount of the stored energy stored by the energy storage component 214. For example, the processor 200 may execute the energy monitor 204 (e.g., computer-executable instructions) to determine a value representing the amount of stored energy. The processor 200 may represent a processor of the device 102 or a processor of the object 104 including the device 102, as described herein.

At 906, the processor 200 may adjust a frequency at which the temperature sensor 108 periodically senses the temperature of the object 104 by analyzing the amount of the stored energy. For example, the processor 200 may execute the temperature sensing manager 202 (e.g., computer-executable instructions) to adjust the temperature-sensing frequency at block 906. The temperature-sensing frequency may be adjusted at block 906 by increasing or decreasing the temperature-sensing frequency, and the analyzing of the amount of stored energy may be based on rules, logic, a model, etc., such as by determining whether the amount of stored energy satisfies a threshold(s). For example, the temperature-sensing frequency may be increased from once every hour to once every half hour if energy reserves are relatively high, or decreased from once every hour to once every two hours if energy reserves are relatively low. These are merely example ways that the temperature-sensing frequency might be adjusted, and the adjustment may vary by implementation.

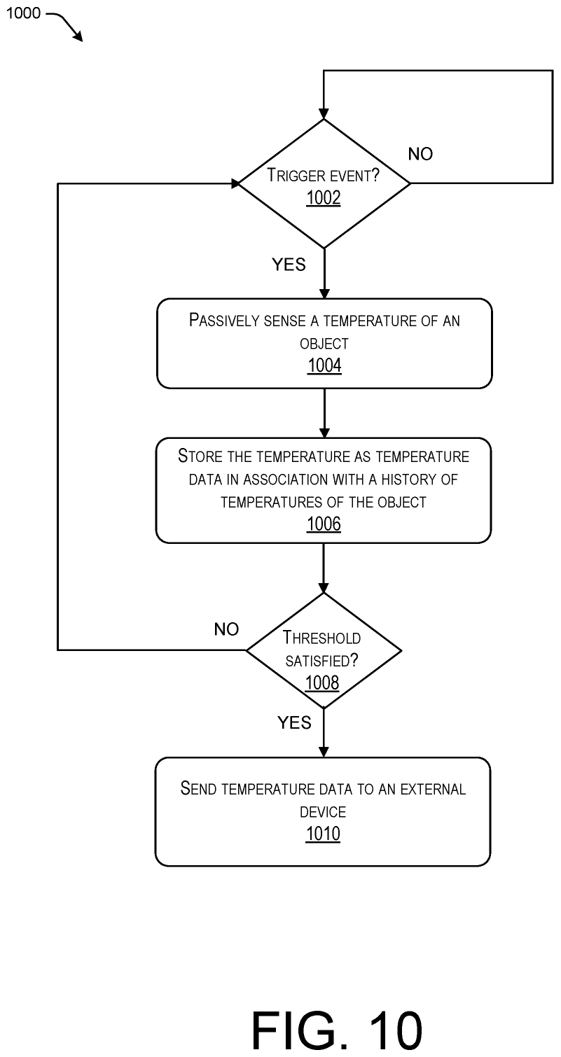

FIG. 10 illustrates an example process 1000 for passively logging the temperature of an object 104 and sending corresponding temperature data 114 to an external device 118 in an energy-efficient manner, according to the techniques described herein. The process 1000 may be implemented by a device 102 coupled to an object 104, by an object 104 including the device 102, or by another suitable device or system. For discussion purposes, the process 1000 is described with reference to the previous figures.

At 1002, a processor 200 may monitor for the occurrence of a trigger event. The processor 200 may represent a processor of the device 102 or a processor of the object 104 including the device 102, as described herein. The object 104 may be any suitable type of object, as described herein, such as a medical device (e.g., a set of electrodes 104(1), an external defibrillator 104(2), etc.). In some examples, the occurrence of the trigger event at block 1002 is the receipt, by a device 102 (or by an object 104 including the device 102), of an interrogation signal from an external device 118. For example, the object 104 may represent a set of electrodes 104(1), and the process 1000 may be triggered by the set of electrodes 104(1) receiving an interrogation signal from an external device 118, such as a case 118(1) in which an external defibrillator 118(2) is stored, an external defibrillator 118(2), or the like. In some examples, the occurrence of the trigger event at block 1002 is a lapse of a period of time (e.g., a lapse of a period of time between sequential temperature measurements). That is, in some examples, the temperature sensor 108 is configured to periodically sense the temperature of an object 104 to which the device 102 is coupled, and, as such, the passage of a period of time my trigger the process 1000 at block 1002. Whatever the trigger event, if the trigger event does not occur, the process 1000 may follow the NO route from block 1002 to continue monitoring for the occurrence of the trigger event, yet conserving energy by refraining from performing temperature sensing and logging until the trigger event occurs. If the trigger event occurs, however, the process 1000 may follow the YES route from block 1002 to block 1004.

At 1004, a temperature sensor 108 may passively sense a temperature of the object 104. The temperature sensor 108 may represent a temperature sensor of the device 102 or a temperature sensor of the object 104 including the device 102, as described herein. In some examples, the operations performed at block 1004 are similar to those performed at block 802 of the process 800, as described above.

At 1006, the temperature may be stored in a memory 110 as temperature data 114 representing a history 116 of temperatures sensed by the temperature sensor 108. The memory 110 may represent a memory of the device 102 or a memory of the object 104 including the device 102, as described herein. In some examples, the operations performed at block 1006 are similar to those performed at block 810 of the process 800, as described above.

At 1008, the processor 200 may determine whether a threshold is satisfied by one or more of the temperatures in the history 116 of temperatures. For example, the process 200 may execute the temperature analyzer 206 (e.g., computer-executable instructions) to compare an individual temperature, multiple temperatures (e.g., a series of sequential temperatures), a statistic computed from the temperatures in the history 116 (e.g., an average temperature), or the like to a threshold temperature. As used herein, a threshold may be “satisfied” if an individual value (e.g., temperature) meets or exceeds the threshold or if the individual value strictly exceeds the threshold. If the threshold is not satisfied, the process 1000 may follow the NO route from block 1008 to block 1002 to monitor for the occurrence of another trigger event, yet conserving energy by refraining from transmitting temperature data 114 until one or more of the temperatures in the history 116 of temperatures satisfies the threshold. In other words, if the temperature does not change from a previous measurement and/or stays within a prescribed temperature range, there may not be anything noteworthy to report to an external device 118 about the temperature of the object 104. On the other hand, if the threshold is satisfied, the process 1000 may follow the YES route from block 1008 to block 1010, where temperature data 114 is sent, via a transceiver 226, to an external device 118 in response to one or more of the temperatures in the history 116 of temperatures satisfying the threshold. In this manner, the device 102 (or the object 104 including the device 102) is configured to wait until there is something noteworthy to before using energy to transmit the temperature data 114 at block 1010, thereby conserving energy.

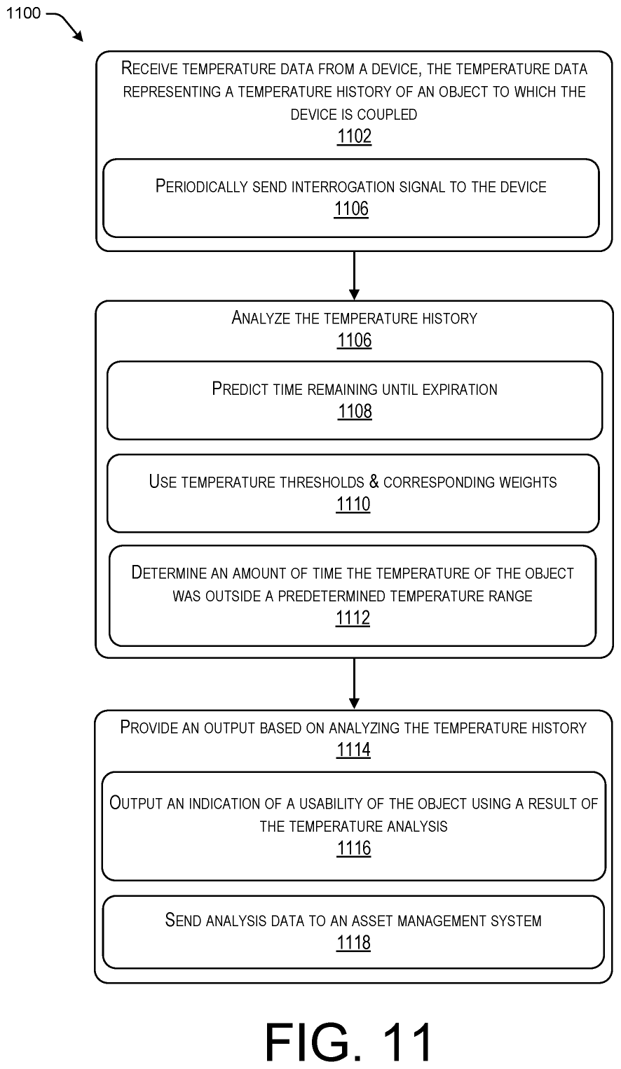

FIG. 11 illustrates an example process 1100 for analyzing a temperature history 116 of an object 104 and providing an output based on the temperature history analysis, according to the techniques described herein. The process 1100 may be implemented by an external device 118 that is external to an object 104 and/or external to a device 102 coupled to the object 104, by a remote system 120, or by another suitable device or system. For discussion purposes, the process 1100 is described with reference to the previous figures.

At 1102, a processor 500 may receive, from a device 102 coupled to an object 104, temperature data 114 representing a temperature history 116 of the object 104. The object 104 may be any suitable type of object, as described herein, such as a medical device (e.g., a set of electrodes 104(1), an external defibrillator 104(2), etc.).

At 1104, the processor 500 may cause the external device 118 to periodically send, to the device 102, interrogation signals to receive updated temperature data 114 representing the temperature history 116 of the object 104. For example, the processor 500 may execute the interrogator 504 (e.g., computer-executable instructions) to periodically interrogate the device 102. Accordingly, the temperature data 114 received at block 1102 may be received in response to an interrogation signal sent to the device 102.

At 1106, the processor 500 may analyze the temperature history 116. For example, the processor 500 may execute the temperature analyzer 506 (e.g., computer-executable instructions) to analyze the temperature history 116. The temperature history 116 can be analyzed in various ways, as indicated by sub-blocks 1108 to 1112.

At 1108, for example, the processor 500 may predict a time remaining until the object 104 is expired. In some examples, the processor 500 may execute the prediction component 508 (e.g., computer-executable instructions) to predict the time remaining until expiration using a result of analyzing the temperature history 116.

At 1110, the processor 500 may use multiple different temperature thresholds and corresponding weights 510 to analyze the temperature history 116 at block 1106, such as to predict the time remaining until expiration of the object 104. For example, the processor 500 may execute the temperature analyzer 506 to determine whether a subset of sequential temperatures in the temperature history 116 satisfy (e.g., exceed) one or more of the different thresholds. For example, the multiple different thresholds may include a first threshold, a second threshold, and so on, where the thresholds are progressively increasing such that the second threshold is greater than the first threshold, and so on. If a subset of sequential temperatures in the temperature history 116 satisfy (e.g., exceed) the first threshold but not the second threshold (e.g., the sequential temperatures fall between the first and second thresholds), the processor 500 may determine an integral associated with the subset of sequential temperatures and may use the integral and a first weight associated with the first temperature threshold to determine a value. This value may be used to predict the time remaining until expiration of the object 104. If, on the other hand, the subset of sequential temperatures in the temperature history 116 satisfy (e.g., exceed) the first threshold and the second threshold, a second weight associated with the second threshold may be used for predicting the remaining life of the object 104. That is, if both thresholds are satisfied, the processor 500 may determine an integral associated with the subset of sequential temperatures and may use the integral and the second weight associated with the second, greater temperature threshold to determine a value. This value may then be used to predict the time remaining until expiration of the object 104.

At 1112, the processor 500 may determine, by analyzing the temperature history 116, an amount of time that a temperature of the object 104 was outside of a predetermined temperature range. The examples of sub-blocks 1108, 1110, and 1112 are merely example ways in which the temperature history 116 can be analyzed. For example, the analyzing of the temperature history 116 may include determining whether a subset of the temperatures in the history 116 of temperatures is less than a threshold. This may be relevant in a situation where the object 104 may be rendered inoperable in temperatures that are below the threshold.

At 1114, an output may be provided via an output device 514 based on analyzing the temperature history at block 1106. The output device(s) 514 can be any suitable type of output device described herein, such as a display 516, a light emitting element 518, a speaker 522, a transceiver 526, or the like.

At 1116, providing the output may include outputting an indication of a usability of the object 104 using a result of analyzing the temperature history 116. In some examples, the usability indication may be output at block 1116 as a warning or a notification that the object 104 should be discarded. In some examples, the usability indication may be output at block 1116 as an indication of a time remaining until the object 104 is expired, an indication that a temperature of the object 104 was outside of a predetermined temperature range (or an indication of an amount of time that the temperature of the object 104 was outside of the range), or the like.

At 1118, providing the output may include sending analysis data to a remote system 120 (e.g., an asset management system). For example, the external device 118 may send an amount of time that the temperature of the object 104 was outside of a predetermined temperature range to an asset management system configured to manage a fleet of objects, wherein the object 104 is one of the objects in the fleet.

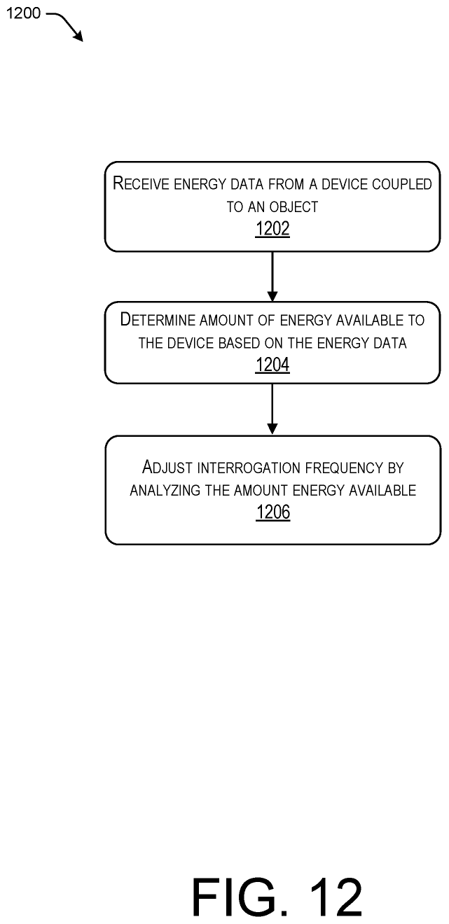

FIG. 12 illustrates an example process 1200 for adjusting an interrogation frequency of an external device 118 based on an amount of energy available to a passive temperature-logging device 102, according to the techniques described herein. The process 1200 may be implemented by an external device 118 that is external to an object 104 and/or external to a device 102 coupled to the object 104, by a remote system 120, or by another suitable device or system. For discussion purposes, the process 1200 is described with reference to the previous figures.

At 1202, a processor 500 may receive, from a device 102 coupled to an object 104, energy data indicating an amount of energy available to the device 102. The object 104 may be any suitable type of object, as described herein, such as a medical device (e.g., a set of electrodes 104(1), an external defibrillator 104(2), etc.).

At 1204, the processor 500 may determine the amount of energy available to the device 102 based on the energy data.

At 1206, the processor 500 may adjust, based on the amount of the energy available, a frequency at which the external device 118 periodically sends interrogation signals to the device 102 to receive updated temperature data representing the temperature history 116 of the object 104. For example, the processor 500 may execute the interrogator 504 (e.g., computer-executable instructions) to adjust the interrogation frequency at block 1206. The interrogation frequency may be adjusted at block 1206 by increasing or decreasing the interrogation frequency, and the analyzing of the amount of available energy may be based on rules, logic, a model, etc., such as by determining whether the amount of available energy satisfies a threshold(s). For example, the interrogation frequency may be increased from once every hour to once every half hour if energy reserves are relatively high, or decreased from once every hour to once every two hours if energy reserves are relatively low. These are merely example ways that the interrogation frequency might be adjusted, and the adjustment may vary by implementation.

### Example Clauses

1. A set of electrodes for an external defibrillator, the set of electrodes comprising: a temperature sensor configured to sense a temperature of the set of electrodes using energy harvested from a non-battery energy source; a memory configured to store temperature data representing a history of temperatures sensed by the temperature sensor, wherein the temperature is one of the temperatures; and a transceiver configured to send the temperature data to an external device.
2. The set of electrodes of clause 1, further comprising an energy harvesting component configured to harvest the energy from the non-battery energy source when the set of electrodes is separated from the external defibrillator, wherein the temperature sensor, the memory, and the transceiver are configured to be powered by the energy.
3. The set of electrodes of clause 2, further comprising: an energy storage component configured to store the energy as stored energy; and a processor configured to: determine an amount of the stored energy; and adjust a frequency at which the temperature sensor periodically senses the temperature of the set of electrodes by analyzing the amount of the stored energy.
4. The set of electrodes of clause 2 or 3, wherein: the electrodes are stored in a container separate from the external defibrillator; and the energy harvesting component is configured to harvest the energy from the non-battery energy source when the electrodes are stored in the container.
5. The set of electrodes of any one of clauses 1 to 4, wherein the transceiver is configured to send the temperature data to the external device in response to one of the temperatures in the history of temperatures satisfying a threshold.
6. The set of electrodes of any one of clauses 1 to 5, wherein: the external defibrillator is stored in a case; and the temperature sensor is configured to sense the temperature of the set of electrodes in response to an interrogation signal received from the external defibrillator or the case.
7. The set of electrodes of any one of clauses 1 to 6, wherein the temperature sensor is configured to periodically sense the temperature of the set of electrodes.
8. The set of electrodes of any one of clauses 1 to 7, wherein the external device is the external defibrillator.
9. An external defibrillator comprising: a temperature sensor configured to sense a temperature of the external defibrillator using energy harvested from a non-battery energy source; a memory configured to store temperature data representing a history of temperatures sensed by the temperature sensor, wherein the temperature is one of the temperatures; and an output device configured to provide an output based on the temperature data.
10. The external defibrillator of clause 9, further comprising an energy harvesting component configured to harvest the energy from the non-battery energy source when the external defibrillator is separated from mains electricity, wherein the temperature sensor and the memory are configured to be powered by the energy.
11. The external defibrillator of clause 10, further comprising: an energy storage component configured to store the energy as stored energy; and a processor configured to: determine an amount of the stored energy; and adjust a frequency at which the temperature sensor periodically senses the temperature of the external defibrillator by analyzing the amount of the stored energy.
12. The external defibrillator of any one of clauses 9 to 11, wherein the output device is configured to output an indication of a usability of the external defibrillator by analyzing the temperature data.
13. The external defibrillator of clause 12, wherein the analyzing of the temperature data comprises determining whether a subset of the temperatures in the history of temperatures is less than a threshold.
14. The external defibrillator of any one of clauses 9 to 13, wherein the temperature sensor is configured to periodically sense the temperature of the external defibrillator.
15. The external defibrillator of any one of clauses 9 to 14, wherein: the output device comprises a transceiver; and providing the output comprises sending, to a server remote from the external defibrillator, the temperature data or a result determined by analyzing the temperature data.
16. An external defibrillator comprising: a processor; and memory storing computer-executable instructions that, when executed by the processor, cause the external defibrillator to: receive, from a device coupled to a set of electrodes, temperature data representing a temperature history of the set of electrodes; analyze the temperature history; predict a time remaining until the set of electrodes is expired using a result of analyzing the temperature history; and output an indication of the time remaining via an output device of the external defibrillator.
17. The external defibrillator of clause 16, wherein multiple different temperature thresholds and corresponding weights are used to analyze the temperature history.
18. The external defibrillator of clause 16 or 17, wherein: analyzing the temperature history comprises: determining that a subset of sequential temperatures in the temperature history exceed a first threshold; determining an integral associated with the subset of sequential temperatures; and determining a value using the integral and a first weight associated with the first threshold; and predicting the time remaining comprises predicting the time remaining using the value.
19. The external defibrillator of any one of clauses 16 to 18, wherein: analyzing the temperature history comprises: determining that a subset of sequential temperatures in the temperature history exceed a first threshold and a second threshold, the second threshold being greater than the first threshold; determining an integral associated with the subset of sequential temperatures; and determining a value using the integral and a second weight associated with the second threshold; and predicting the time remaining comprises predicting the time remaining using the value.
20. The external defibrillator of any one of clause 16 to 19, wherein the computer-executable instructions, when executed by the processor, further cause the external defibrillator to periodically send, to the device, interrogation signals to receive updated temperature data representing the temperature history of the set of electrodes.
21. The external defibrillator of any one of clauses 16 to 20, wherein the computer-executable instructions, when executed by the processor, further cause the external defibrillator to: receive, from the device, energy data indicating an amount of energy available to the device; and adjust, based on the amount of the energy available, a frequency at which the external defibrillator periodically sends interrogation signals to the device to receive updated temperature data representing the temperature history of the set of electrodes.
22. A device configured to be coupled to an object, the device comprising: a temperature sensor configured to sense a temperature of the object using energy harvested from a non-battery energy source; a memory configured to store temperature data representing a history of temperatures sensed by the temperature sensor, wherein the temperature is one of the temperatures; and an output device configured to provide an output based on the temperature data.
23. The device of clause 22, further comprising an energy harvesting component configured to harvest the energy from the non-battery energy source when the object is separated from mains electricity, wherein the temperature sensor and the memory are configured to be powered by the energy.
24. The device of clause 23, further comprising: an energy storage component configured to store the energy as stored energy; and a processor configured to: determine an amount of the stored energy; and adjust a frequency at which the temperature sensor periodically senses the temperature of the object by analyzing the amount of the stored energy.
25. The device of any one of clauses 22 to 24, wherein: the output device comprises a transceiver; and providing the output comprises sending the temperature data to an external device external to the object.
26. The device of clause 25, wherein the transceiver is configured to send the temperature data to the external device in response to one of the temperatures in the history of temperatures satisfying a threshold.
27. The device of any one of clauses 22 to 26, wherein the temperature sensor is configured to sense the temperature of the object in response to an interrogation signal received from an external device.
28. An external device external to an object, the external device comprising: a processor; and memory storing computer-executable instructions that, when executed by the processor, cause the external device to: receive, from a device coupled to the object, temperature data representing a temperature history of the object; determine, by analyzing the temperature history, an amount of time that a temperature of the object was outside of a predetermined temperature range; and send the amount of time to an asset management system configured to manage a fleet of objects, wherein the object is one of the objects in the fleet.
29. The external device of clause 28, wherein the object comprises an external defibrillator.
30. The external device of clause 28 or 29, wherein the object comprises a set of electrodes for an external defibrillator.
31. A method comprising: sensing, by a temperature sensor of a device that is coupled to an object, a temperature of the object, wherein the temperature sensor senses the temperature of the object using energy harvested from a non-battery energy source; storing, in a memory of the device as temperature data, the temperature in association with a history of temperatures of the object that were previously sensed by the temperature sensor; and providing, by the device, an output based on the temperature data.
32. The method of clause 31, further comprising: harvesting, by the device, the energy from the non-battery energy source when the object is separated from mains electricity; and supplying power to the temperature sensor and to the memory using the energy.
33. A method comprising: receiving, by an external device external to an object, from a device coupled to the object, temperature data representing a temperature history of the object; analyzing, by the external device, the temperature history; and outputting, by the external device, using a result of the analyzing of the temperature history, an indication of a usability of the object.
34. A medical device comprising: a temperature sensor configured to sense a temperature of the medical device using energy harvested from a non-battery energy source; a memory configured to store temperature data representing a history of temperatures sensed by the temperature sensor, wherein the temperature is one of the temperatures; and an output device configured to provide an output based on the temperature data.
35. The medical device of clause 34, further comprising an energy harvesting component configured to harvest the energy from the non-battery energy source when the medical device is separated from mains electricity, wherein the temperature sensor and the memory are configured to be powered by the energy.
36. A set of electrodes for an external defibrillator, the set of electrodes comprising: a temperature sensor configured to sense a temperature of the set of electrodes by detecting a change in voltage across a diode; a memory configured to store temperature data representing a history of temperatures sensed by the temperature sensor, wherein the temperature is one of the temperatures; and a transceiver configured to send the temperature data to an external device.

While the example clauses described above are described with respect to one particular implementation, it should be understood that, in the context of this document, the content of the example clauses can also be implemented via a method, device, system, computer-readable medium, and/or another implementation. Additionally, any one of examples 1-36 may be implemented alone or in combination with any other of the examples 1-36.

### CONCLUSION

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All processes described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain implementations are described herein, including the best mode known to the inventors for carrying out implementations of the disclosure. Of course, variations on these described implementations will become apparent to those of ordinary skill in the art upon reading the foregoing description. The inventors expect skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

1. A set of electrodes for an external defibrillator, the set of electrodes comprising:
a temperature sensor configured to sense a temperature of the set of electrodes using energy harvested from a non-battery energy source;
a memory configured to store temperature data representing a history of temperatures sensed by the temperature sensor, wherein the temperature is one of the temperatures; and
a transceiver configured to send the temperature data to an external device.

2. The set of electrodes of claim 1, further comprising an energy harvesting component configured to harvest the energy from the non-battery energy source when the set of electrodes is separated from the external defibrillator, wherein the temperature sensor, the memory, and the transceiver are configured to be powered by the energy.

3. The set of electrodes of claim 2, further comprising:
an energy storage component configured to store the energy as stored energy; and
a processor configured to:
determine an amount of the stored energy; and
adjust a frequency at which the temperature sensor periodically senses the temperature of the set of electrodes by analyzing the amount of the stored energy.

4. The set of electrodes of claim 2, wherein:
the set of electrodes are stored in a container separate from the external defibrillator; and
the energy harvesting component is configured to harvest the energy from the non-battery energy source when the set of electrodes are stored in the container.

5. The set of electrodes of claim 1, wherein the transceiver is configured to send the temperature data to the external device in response to one of the temperatures in the history of temperatures satisfying a threshold.

6. The set of electrodes of claim 1, wherein:
the external defibrillator is stored in a case; and
the temperature sensor is configured to sense the temperature of the set of electrodes in response to an interrogation signal received from the external defibrillator or the case.

7. The set of electrodes of claim 1, wherein the temperature sensor is configured to periodically sense the temperature of the set of electrodes.

8. The set of electrodes of claim 1, wherein the external device is the external defibrillator.

9. An external defibrillator comprising:
a temperature sensor configured to sense a temperature of the external defibrillator using energy harvested from a non-battery energy source;
a memory configured to store temperature data representing a history of temperatures sensed by the temperature sensor, wherein the temperature is one of the temperatures; and
an output device configured to provide an output based on the temperature data.

10. The external defibrillator of claim 9, further comprising an energy harvesting component configured to harvest the energy from the non-battery energy source when the external defibrillator is separated from mains electricity, wherein the temperature sensor and the memory are configured to be powered by the energy.

11. The external defibrillator of claim 10, further comprising:
an energy storage component configured to store the energy as stored energy; and
a processor configured to:
determine an amount of the stored energy; and
adjust a frequency at which the temperature sensor periodically senses the temperature of the external defibrillator by analyzing the amount of the stored energy.

12. The external defibrillator of claim 9, wherein the output device is configured to output an indication of a usability of the external defibrillator by analyzing the temperature data.

13. The external defibrillator of claim 12, wherein the analyzing of the temperature data comprises determining whether a subset of the temperatures in the history of temperatures is less than a threshold.

14. The external defibrillator of claim 9, wherein the temperature sensor is configured to periodically sense the temperature of the external defibrillator.

15. The external defibrillator of claim 9, wherein:
the output device comprises a transceiver; and
providing the output comprises sending, to a server remote from the external defibrillator, the temperature data or a result determined by analyzing the temperature data.

16. The external defibrillator of claim 15, wherein the transceiver is configured to send the temperature data to the server in response to one of the temperatures in the history of temperatures satisfying a threshold.

17. The external defibrillator of claim 9, wherein:
the external defibrillator is stored in a case; and
the temperature sensor is configured to sense the temperature of the external defibrillator in response to an interrogation signal received from the case.

18. A method comprising:
sensing, by a temperature sensor of a device that is coupled to an object, a temperature of the object, wherein the temperature sensor senses the temperature of the object using energy harvested from a non-battery energy source;
storing, in a memory of the device as temperature data, the temperature in association with a history of temperatures of the object that were previously sensed by the temperature sensor; and
providing, by the device, an output based on the temperature data.

19. The method of claim 18, further comprising:
harvesting, by the device, the energy from the non-battery energy source when the object is separated from mains electricity; and
supplying power to the temperature sensor and to the memory using the energy.

20. The method of claim 19, further comprising:
storing, by the device, the energy as stored energy;
determining an amount of the stored energy; and
adjusting a frequency at which the temperature sensor periodically senses the temperature of the object by analyzing the amount of the stored energy.
